(19) United States
(12) Patent Application Publication
(10) Pub. No.: US 2016/0206631 A1
(43) Pub. Date: Jul. 21, 2016

(54) OXYSTEROLS THAT ACTIVATE LIVER X RECEPTOR SIGNALING AND INHIBIT HEDGEHOG SIGNALING

(71) Applicant: THE REGENTS OF THE UNIVERSITY OF CALIFORNIA, OAKLAND, CA (US)

(72) Inventors: Farhad PARHAMI, Los Angeles, CA (US); Michael JUNG, Los Angeles, CA (US); Khanhlinh NGUYEN, Los Angeles, CA (US); Dongwon YOO, Los Angeles, CA (US); William MATSUI, Baltimore, MD (US)

(73) Assignees: JOHNS HOPKINS UNIVERSITY, BALTIMORE, MD (US); THE REGENTS OF THE UNIVERSITY OF CALIFORNIA, OAKLAND, CA (US)

(21) Appl. No.: 14/945,133

(22) Filed: Nov. 18, 2015

Related U.S. Application Data
(63) Continuation of application No. 13/579,278, filed on Aug. 15, 2012, now abandoned.
(60) Provisional application No. 61/305,046, filed on Feb. 16, 2010.

(51) Int. Cl. A61K 31/575; A61K 31/58; A61K 45/06

(52) U.S. Cl. CPC A61K 31/575; A61K 45/06; A61K 31/58

(57) ABSTRACT

This invention relates, e.g., to compositions comprising oxysterol compounds represented by Formula I or Formula II, e.g., comprising one or more of Oxy 16, Oxy 22, Oxy30, Oxy 31, Oxy35, Oxy37, Oxy43, Oxy44, Oxy45 or Oxy47. The compounds are shown to be Hedgehog pathway inhibiting, and to act as agonists for liver X receptor (LXR). Also disclosed are methods of using compositions of the invention to inhibit Hedgehog signaling effects, such as cell proliferation, including treating subjects in need thereof, and pharmaceutical compositions and kits for implementing methods of the invention.

This application claims the benefit of the filing date of U.S. Provisional application 61/305,046, filed Feb. 16, 2010, which is incorporated by reference herein in its entirety.

This invention were made with Government support under Grant No. AR050426 awarded by the National Institutes of Health. The government has certain rights in this invention.

### BACKGROUND INFORMATION

Hedgehog molecules have been shown to play key roles in a variety of physiological processes including tissue patterning, mitogenesis, morphogenesis, cellular differentiation, differentiation of stein cells into mature cells, embryonic development, cardiovascular disease, bone formation, and cancer (1-7). In addition to its role in embryonic development, Hedgehog (Hh) signaling plays a crucial role in postnatal development and maintenance of tissue/organ integrity and function (8-14). Studies using genetically engineered mice have demonstrated that Hedgehog signaling is important during skeletogenesis as well as in the development of osteoblasts in vitro and in vivo (15-18). Aberrant Hh signaling has been implicated in various cancers including hereditary forms of medulloblastoma, basal cell carcinoma, multiple myeloma, acute lymphoblastic leukemia, and prostate, breast, colon, and lung cancers, (1, 4, 19, 20).

Hedgehog signaling involves a very complex network of signaling molecules that includes plasma membrane proteins, kinases, phosphatases, and factors that facilitate the shuffling and distribution of Hedgehog molecules (21-23). Production of Hedgehog molecules from a subset of producing/signaling cells involves its synthesis, autoprocessing, and lipid modification (24, 25). Lipid modification of Hedgehog, which appears to be essential for its functionality, involves the addition of a cholesterol molecule to the C-terminal domain of the auto-cleaved Hedgehog molecule and palmitoylation at its N-terminal domain. Additional accessory factors help shuttle Hedgehog molecules to the plasma membrane of the signaling cells, release them into the extracellular environment, and transport them to the responding cells.

Hedgehog signaling can promote cell division and proliferation of cells, e.g., cancerous and tumorous cells; and dysregulated (aberrant) Hedgehog signaling has been implicated in the proliferation and/or metastasis of a variety of cancers including, e.g., basal cell carcinoma, melanoma, multiple myeloma, leukemia, stomach cancer, pancreatic cancer, bladder cancer, prostate cancer, ovarian cancer, and bone cancer, such as osteosarcoma (26-32). Therefore, the inhibition of Hedgehog signaling might offer a route for treating, e.g., certain cancers.

Liver X receptors (LXRs) are members of the family of nuclear hormone receptors. They are involved in a variety of physiologic processes including lipid and glucose metabolism, cholesterol homeostasis, and anti-inflammatory signaling (33-36). Two isoforms of LXR have been identified and are referred to as LXRα and LXRβ. Liver X receptors have been shown (e.g., by the present inventors in co-pending U.S. application Ser. No. 12/374,296, filed Jan. 16, 2009) to be activated by certain naturally occurring oxysterols. Physiologic ligands for LXRs include naturally occurring oxysterols. LXRs appear to play a role in growth and progression of various tumor cells including breast, prostate, and ovarian (37-39). As such, LXRs may serve as therapeutic targets for various disorders including cancer, atherosclerosis, diabetes, and Alzheimer's disease (40-43).

### DESCRIPTION OF THE DRAWINGS

FIG. 1 shows Expression of LXR isoforms in osteosarcoma cells. Saos-2 cells were cultured in DMEM containing 10% fetal bovine serum (FBS) until confluent. mRNA expression for LXRα and LXRβ was quantified by Q-RT-PCR and normalized to GAPDH. Data from a representative experiment are reported as the mean of triplicate determinations±SD relative to the expression level of LXRα (p<0.001 for LXRα vs. LXRβ expression).

FIG. 2 shows Expression of LXR target genes in osteosarcoma cells. Saos-2 cells were treated with control vehicle or TO901317 (TO) LXR ligand for 72 hours. mRNA expression for LXR target genes ABCA1 and SREBP1c was quantified by Q-RT-PCR and normalized to GAPDH. Data from a representative experiment are reported as the Data from a representative experiment are reported as the mean of triplicate determinations±SD relative to the expression level of LXRα (p<0.001 for control vs. both concentrations of TO for ABCA1 and SREBP1c mRNA).

FIG. 3 shows that Oxy16 is a synthetic oxysterol that activates LXR signaling. Preliminary studies with Oxy16 has demonstrated strong induction of LXR target genes ABCA1 and ABCG1, but not SREBP1c, in osteosarcoma cells.

FIG. 4 shows that LXR ligands inhibit clonogenic growth of human osteosarcoma cells. Saos-2 and U2O2 cells were treated with control vehicle, or 1 μM of TO901317 (TO), 22(R)-hydroxycholesterol, or Oxy16 for 72 hours. Next, cells were harvested and examined for clonogenic growth in non-adherent plates after 10 days of culturing. Data from a representative of two separate experiments are reported as the relative number of colonies formed by cells treated with LXR ligands relative to cells treated with control vehicle (% of control).

FIG. 5 shows the effect of TO901317 (TO) and cyclopamine (Cyc) on Ptch1 expression in osteosarcoma cells. Saos-2 cells were cultured in medium containing 2% FBS and were treated at confluence with 4 μM Cyc, 2 or 4 τM TO, alone or in combination for 72 hours. Expression of Ptch1 and Gli1 (data not shown) mRNA was measured by Q-RT-PCR and normalized to GAPDH. Data from a representative experiment are reported as the mean of triplicate determinations±SD (p<0.001 for Control vs. all other treatment groups).

FIG. 6 shows the effect of LXR ligands on human multiple myeloma cells. NCI-11929 multiple myeloma cells were treated for 96 hours with control vehicle or 1 μM of each compound as shown. Next, drugs were removed and cells were plated in methylcellulose. Clonogenic growth of colonies determined after 10 days. Data are reported as percentage of colony number normalized to control group.

FIG. 7 shows the effect of LXR ligands on prevalence of stem cells in multiple myeloma cell cultures. NCI-H929 multiple myeloma cells were treated for 96 hours with control vehicle or 1 μM of each compound as shown. Next, percentage of CD138negative cells in the same number of starting cells from each group was determined by flow cytometry.

FIG. 8 shows the effect of LXR ligands on prevalence of stem cells in multiple myeloma cell cultures. NCI-H929 multiple myeloma cells were treated for 96 hours with control vehicle or 1 μM of each compound as shown. Next, percentage of ALDH+ cells in the same number of starting cells from each group was determined by flow cytometry.

FIG. 9 shows hedgehog expression by human pancreatic cancer cells. Expression of Shh and Ihh mRNA in human cultures of pancreatic cancer cells, CAPAN-1, L3.6p1, and E3LZ10.7 were analyzed by Q-RT-PCR and normalized to GAPDH expression. Cells were cultured in DMEM containing 10% FBS and RNA was extracted 3 days after seeding. Data from a representative experiment are reported as the mean of triplicate determinations±SD (p<0.001 for CAPAN-1 vs. other two cell types for Shh and 11 h mRNA expression).

FIG. 10 shows inhibition of pancreatic cancer cell induced Hedgehog signaling by LXR agonists. C3H10T1/2 cells were pretreated for 2 hours with control vehicle or the LXR agonists TO901317 (TO, 2 μM) or Oxy16 (5 μM), or the Hedgehog pathway inhibitor cyclopamine (Cyc, 4 μM). Next, cells were treated with DMEM containing 5% PBS or CAPAN-1 CM in the presence or absence of TO, Oxy16, or Cyc. After 48 hours, RNA was extracted and analyzed by Q-RT-PCR for the expression of Hh target genes Ptch1, HHIP, and Gli1 and normalized to GAPDH expression. Data from a representative experiment are reported as the mean of triplicate determinations±SD (p<0.001 for Control vs. CM and for CM vs. CM+TO, CM+Cyc, and CM+Oxy16 for Ptch1, HHIP, and Gli1 expression).

FIG. 11 shows inhibition of pancreatic cancer cell-induced alkaline phosphatase activity by LXR agonists. C3H10T1/2 cells were pretreated for 2 hours with control vehicle or the LXR agonists TO901317 (TO, 2 μM) or Oxy16 (5 μM), or the Hedgehog pathway inhibitor cyclopamine (Cyc, 4 μM). Next, cells were treated with DMEM containing 5% FBS or CAPAN-1 CM in the presence or absence of TO, Oxy16, or Cyc. After 3 days, alkaline phosphatase activity assay using whole cell lysates was performed. Results from a representative experiment are reported as the mean of quadruplicate determinations±SD (p<0.001 for Control vs. CM and for CM vs. CM+TO, CM+Cyc, and CM+Oxy16).

### DESCRIPTION

The present inventors identify herein a group of synthetic oxysterols that are agonists or ligands of a liver X receptor (LXR), and that can inhibit Hedghog (Hh) signaling. Furthermore, these oxysterols are shown to inhibit clonogenic growth of human cancer cells, and thus to be useful as therapeutic agents to treat conditions mediated by excess cell proliferation, such as cancers. In addition, LXR signaling induced by these oxysterols (or by TO901317) is shown to inhibit the induction of Hh signaling in stromal/fibroblastic cells by human pancreatic cancer cells that express Hh proteins. For example, the Examples herein show the inhibition by oxysterols of the invention of cell growth of the human osteosarcoma cells Saos-2 and U2OS, which are art-recognized models for studying human solid bone tumors. Other cell lines tested include the pancreatic cancer cell lines, Capan-1, E3LZ10.7, and L3.6p1, multiple myeloma cells, and human acute lymphocytic leukemia (ALL) cells. Surprisingly, only a subset of the synthetic oxysterols that were tested exhibited this behavior.

This invention relates, e.g., to a composition comprising a compound represented by Formula I. In one embodiment of the invention, the composition comprises one or more of the Oxysterols, Oxy30, Oxy35, Oxy37, Oxy43, Oxy44, Oxy45 or Oxy47. The structures of these compounds are shown in Example I.

Another aspect of the invention is a composition comprising a compound represented by Formula II. In one embodiment of the invention, the composition comprises one or more of the oxysterols, Oxy 16, Oxy 22, Oxy30, Oxy 31, Oxy35, Oxy37, Oxy43, Oxy44, Oxy45 or Oxy47. A composition comprising a compound represented by Formula II or one or more of Oxy16, Oxy 22, Oxy30, Oxy 31, Oxy35, Oxy37, Oxy43, Oxy44, Oxy45 or Oxy47 may be a pharmaceutical or bioactive composition (e.g. a composition for use in activating LXR, inhibiting Hh activity, or treating LXR-mediated conditions, including conditions characterized by proliferating cells, such as cancers), which comprises, in addition to the compounds, a pharmaceutically active carrier. Compositions comprising the compound represented by Formula II or by one or more of Oxy 16, Oxy 22, Oxy30, Oxy 31, Oxy35, Oxy37, Oxy43, Oxy44, Oxy45 or Oxy47 are sometimes referred to herein as “compositions of the invention.” The structures of these compounds is shown in Example I. Oxysterols that do not exhibit the LXR activation/Hh inhibition activity are not encompassed by Formula II.

Another aspect of the invention is a method for stimulating a liver X receptor (LXR) and/or inhibiting Hedgehog (Hh) signaling (inhibiting a Hh pathway-mediated response) in a cell or tissue, comprising contacting the cell or tissue with an effective amount of a compound of the invention. The contacting may be performed in vitro or in a cell or tissue that is in a subject.

Another aspect of the invention is a method for reducing proliferation or metastatic activity of a cell, comprising contacting the cell with an effective amount of a composition of the invention. In embodiments of the invention, the cell is in vitro, or is in a subject; the cell is a benign tumor cell; or the cell is a cancer cell (e.g., a basal cell carcinoma cell, medulloblastoma cell, small cell lung cancer cell, pancreatic cancer cell, stomach cancer cell, pancreatic cancer cell, esophageal cancer cell, colorectal cancer cell, melanoma cell, bladder cancer cell, bone cancer cell, osteosarcoma cell, multiple myeloma cell, ovarian cancer cell, acute or chronic leukemia cell, or a tissue thereof). One embodiment of the invention is a method for treating a subject in need of inhibiting cell proliferation, comprising administering to the subject an effective amount of a composition of the invention. By “metastatic activity” is meant the ability of the cells to metastasize.

Another aspect of the invention is a method for treating a subject having a disease or condition that is mediated by an LXR pathway, comprising administering to the subject an LXR-stimulatory effective amount of a composition of the invention. A variety of such conditions will be evident to a skilled worker. Suitable conditions include, e.g., cardiovascular diseases, Alzheimer's disease, rheumatoid arthritis, osteoarthritis, and other inflammatory conditions.

Another aspect of the invention is a method for treating a subject having a cancer, a cardiovascular disease, Alzheimer's disease, rheumatoid′ arthritis, osteoarthritis, or another inflammatory condition, comprising administering to the subject a therapeutically effective amount of a composition of the invention.

Another aspect of the invention is a method for reducing the prevalence of cancer stem cells in a subject, comprising administering to the subject an effective amount of a composition of the invention. The prevalence of stem cells in a cell population can be reduced by a method of the invention to between about 5 to 35% of total cells, with increments of about 5% included in the range.

Another aspect of the invention is a kit, for carrying out one or more of the methods of the invention, comprising a pharmaceutically effective amount of a composition of the invention, optionally in a container.

In any of the methods or kits of the invention, particularly for treating a subject, a composition of the invention may optionally be in combination with one or more other suitable therapeutic agents, such as a Hedgehog inhibiting LXR agonist and/or another inhibitor of Hh signaling (e.g., a Smoothened antagonist). Any therapeutic agent that is suitable for treatment of a particular condition can be used. Suitable treatments will be evident to one skilled in the art. For example, for treatment of a cancer, a conventional chemotherapeutic drug can be used in combination with a composition of the invention; and for treatment of a cardiovascular or lipid disorder, a statin can be used in combination with a composition of the invention.

As used herein, a liver X receptor (LXR) agonist is a compound that stimulates LXRα, LXRβ, or both. More generally, the term “liver X receptor (LXR)” indicates LXRα, LXRβ, or both. An LXR agonist is a chemical or biological substance that can bind to a receptor and trigger a response in a particular type of cell. A Hedgehog inhibitor is a chemical or biological substance that can reduce or eliminate specific biological or biochemical processes, and “inhibiting” refers to the effect of such substances on such processes in a cell. Treatment of bone marrow stromal cells (MSC) with a composition of the invention can inhibit spontaneous osteogenic differentiation of these cells, as well as inhibiting their activation in response to inducers of Hedgehog pathway signaling.

The experiments discussed herein indicate that the activation of the nuclear hormone receptor, liver X receptor (LXR), by compositions of the invention, can inhibit Hedgehog signaling in a controlled manner. Activation of LXR therefore may offer a route to interfering with dysregulated Hedgehog signaling for the treatment of disease. Without wishing to be bound by any particular mechanism, it is suggested that the inhibition of steps and/or regulators of the Hedgehog pathway through the activation of LXR can serve as a method for inhibiting Hedgehog signaling; and thus such inhibitors can be used to treat diseases and disorders, such as certain cancers, that are mediated by aberrant Hh signaling. However, other mechanisms by which the compositions act to treat the diseases or conditions discussed herein are also encompassed. These include LXR-dependent or -independent mechanisms, and Hh-dependent or -independent mechanisms.

Compositions of the invention can be used to modulate LXR activity and/or Hedgehog signaling in a variety of cell types. For example, in the case of basal cell carcinoma, a topical application of LXR activators can inhibit the increased Hedgehog pathway activity that appears to be a cause of the disease. Another example is the inhibition of medulloblastoma in animal models or in humans, where, again, Hh signaling appears to be causally related to the cancer.

Hedgehog inhibitors of the present invention can be distinguished from some previously described inhibitors, at least because these previously described inhibitors directly target the Hedgehog signaling transducer molecule, Smoothened, on cells that respond to Hedgehog signaling. By contrast, without wishing to be bound by any particular mechanism, it is suggested that the oxysterols of the present invention do not act through inhibition of Smoothened, since a direct activator of Smoothened still activates Hedgehog signaling in the presence of the oxysterols, in contrast to the activation of the pathway by sonic Hedgehog which is inhibited in the presence of LXR activators. Sonic Hedgehog activates the pathway by binding to a receptor, Patched, upstream of Smoothened in the signaling cascade.

Unlike some oxysterols, such as naturally occurring 25-hydroxycholesterol and synthetic Oxy 13 (discussed in U.S. application Ser. No. 12/374,296), which are LXR agonists that leave the Hedgehog pathway active, the oxysterols of the present invention are LXR agonists that have the net effect of inhibiting the Hedgehog pathway. For the treatment of conditions, diseases, or disorders in which aberrant Hedgehog signaling is implicated, the use of Hedgehog-inhibiting LXR agonists of the invention is preferred.

As used herein, the singular forms “a,” “an” and “the” include plural referents unless the context clearly dictates otherwise. For example, “an” agonist includes multiple molecules, e.g. 2, 3, 4, 5 or more agonists, which can be the same or different.

A “subject,” as used herein, includes any animal that exhibits a symptom of a condition that can be treated with a Hedgehog inhibiting LXR agonist of the invention. Suitable subjects (patients) include laboratory animals (such as mouse, rat, rabbit, or guinea pig), farm animals, and domestic animals or pets (such as a cat or dog). Non-human primates and, preferably, human patients, are included. Typical subjects include animals that exhibit aberrant amounts (higher amounts than a “normal” or “healthy” subject) of one or more physiological activities that are stimulated by Hedgehog signaling. The aberrant activities may be regulated by any of a variety of mechanisms, including activation of a Hedgehog activity. The aberrant activities can result in a pathological condition.

An “effective amount,” as used herein, includes an amount that can bring about at least a detectable effect. A “therapeutically effective amount,” as used herein, refers to an amount that can bring about at least a detectable therapeutic response in a subject being treated (e.g. the amelioration of a symptom), over a reasonable time frame. For example, a “therapeutic effect” can refer to a measurable amount of the inhibition of growth of cells causing or contributing to a cell proliferative disorder, or the inhibition of the production of factors (e.g., growth factors) causing or contributing to a cell proliferative or metastatic or inflammatory disorder. A therapeutic effect can relieve to some extent one or more of the symptoms of a cell proliferative or metastatic or inflammatory disorder. A therapeutic effect may refer to one or more of the following: 1) reduction in the number of cancer cells; 2) reduction in tumor size; 3) inhibition (e.g., slowing to some extent, preferably stopping) of cancer cell infiltration into peripheral organs; 4) inhibition (e.g., slowing to some extent, preferably stopping) of tumor metastasis; 5) inhibition, to some extent, of tumor growth; 6) reduction on the number and/or biological activity of cancer stem cells; and/or 7) relieving to some extent one or more of the symptoms associated with an LXR-mediated disorder that is being treated, such as, e.g., inhibition or regression of atherosclerotic lesions, inhibition of Alzheimer's disease, or inhibition of inflammatory responses in arthritis.

In embodiments of the invention, the amount of, e.g., reduction of proliferation or metastatic activity of a cell or tissue, stimulation of an LXR, or inhibition or hedgehog signaling can vary depending upon the particular assay or condition being measured, the amount of the oxysterol administered, etc, and can be routinely determined using conventional methods. For example, the inhibited value can be about 1%, 5%, 10%, 20%, 30%, 40%, 50% or more of that in the untreated sample; and the stimulated value can be about 1%, 5%, 10%, 20%, 30%, 40%, 50% or more of the untreated sample. Intermediate values in these ranges are also included.

A variety of conditions can be treated by compounds of the invention. Among the conditions that can be treated by methods of the invention are cell-proliferative disorders that are mediated by Hedgehog signaling. “Cell proliferative disorders” refer to disorders wherein unwanted cell proliferation of one or more subset(s) of cells in a multicellular organism occurs, resulting in harm (e.g., discomfort or decreased life expectancy) to the multicellular organism. Cell proliferative disorders can occur in a variety of animals, including humans Cell proliferative disorders include cancers. Cancers whose growth and/or metastasis can be inhibited by inhibition of Hedgehog signaling include, e.g., basal cell carcinoma (e.g., using a topical formulation) or other solid tumors, including medulloblastoma, small cell lung cancer, pancreatic cancer, stomach cancer, esophageal cancer, colorectal cancer, ovarian cancer, multiple myeloma, leukemia, prostate cancer and breast cancer (e.g., using a systemic formulation).

Support for the conclusion that the LXR activators of the present invention can inhibit cancer cell growth is provided, e.g., by the following references, which indicate that other LXR activators exhibit this effect:
Vedin L, Lewandowski S A, Parini P, Gustafsson J, Steffensen K R. The oxysterol receptor LXR inhibits proliferation of human breast cancer cells. Carcinogenesis 30:575-579; 2009.
Chuu C, Hiipakka R A, Kokontis J M, Fukuchi J, Chen R, Liao S. Inhibition of tumor growth and progression of LNCaP prostate cancer cells in athymic mice by androgen and liver X receptor agonist. Cancer Res 66:6482-6486; 2006.
Geyeregger R, Shehata M, Zeyda M, Kiefer F W, Stuhlmeier K M, Porpaczy E, Zlabinger G J, Jager U, Stulnig T M. Liver X receptors interfere with cytokine-induced proliferation and cell survival in normal and leukemic lymphocytes. J Leukoc Biol; 2009 [Epub ahead of print].
Scoles D R, Xu X, Wang H, Tran H, Taylor-Harding B, Li A, Karlan B Y. Liver X receptor agonist inhibits proliferation of ovarian carcinoma cells stimulated by oxidized low density lipoprotein. Gynecological Oncology 116:109-116; 2009.

Furthermore, a skilled worker will recognize that a variety of other conditions that are mediated by the LXR pathway can also be treated with a composition of the invention. Such conditions include, e.g., cardiovascular diseases including, but not limited to, arteriosclerosis, angina pectoris, myocardial infarction, and stroke; Alzheimers disease; rheumatoid arthritis; osteoarthritis; and a variety of other inflammatory conditions.

Support for the conclusion that the LXR activators of the present invention can inhibit or prevent atherosclerosis is provided, e.g., in the following references, which indicate that other LXR activators exhibit this effect:
Joseph S B, McKillingin E, Pei L, Watson M A, Collins A R, Laffitte B A, Chen M, Hoh G, Goodman J, Hagger G N, Tran J, Tippin T K, Wang X, Lusis A J, Hsueh W A, Law R E, Collins J L, Willson T M, Tontonoz P. Synthetic LXR ligand inhibits the development of atherosclerosis in mice. Proc Nat Acad Sci 99:7604-7609; 2002.
Naik S U, Wang X, Da Silva J S, Jaye M, Macphee C H, Reilly M P, Billheimer J T, Rothblat G H, Rader D J. Pharmacological activation of liver X receptors promotes reverse cholesterol transport in vivo. Circulation 113:90-97; 2006.
Dacheng P, Hiipakka R A, Dai Q, Gua J, Reardon C A, Getz G S, Liao S. Antiatherosclerotic effects of a. novel synthetic tissue-selective steroidal liver X receptor agonist in low-density lipoprotein receptor-deficient mice. J Pharmacol Exp Ther 327:332-342; 2008.
Fievet C, Staels B. Liver X receptor modulators: effects on lipid metabolism and potential use in the treatment of atherosclerosis. Biochem Pharmacol 77:1316-1327; 2009.
Verschuren L, de Vries-van der Weij J, Zadelaar S, Kleemann R, Kooistra T. LXR agonist suppresses atherosclerotic lesion growth and promotes lesion regression in apoE*3Leiden mice: time course and mechanisms. J Lip Res 50:301-311; 2009.

Support for the conclusion that the LXR activators of the present invention can regulate inflammation is provided, e.g., by the following references, which indicate that other LXR activators exhibit this effect:
Zelcer N, Tontonoz P. Liver X receptors as integrators of metabolic and inflammatory signaling. J Clin Invest 116:607-614; 2006.
Morales J R, Ballesteros I, Denis J M, Hurtado O, Vivancos J, Nombela F, Lizasoain I, Castrillo A, Moro M A. Activation of liver X receptors promotes neuroprotection and reduces brain inflammation in experimental stroke. Circulation 118:1450-1459; 2008.
Korf H, Beken S V, Romano M, Steffensen K R, Stijlemans B, Gustafsson J, Grooten J, Huygen K. Liver X receptors contribute to the protective immune response against Mycobacterium tuberculosis in mice. J Clin Invest 119: 1626-1637; 2009.
Gong H, He J, Lee J H, Mallick E, Gao X, Li S, Homanics G E, Xie W. Activation of the liver X receptor prevents lipopolysaccharide-induced lung injury. J Biol Chem 284:30113-30121; 2009.
Paterniti I, Genovese T, Mazzon E, Crisafulli C, Di Paola R, Galuppo M, Bramanti P, Cuzzocrea S. Liver X receptor agonist treatment regulates inflammatory response after spinal chord trauma.

Support for the conclusion that the LXR activators of the present invention can inhibit or prevent Alzheimer's disease is provided, e.g., by the following references, which indicate that other LXR activators exhibit this effect:
Vaya J, Schipper H M. Oxysterols, cholesterol homeostasis, and Alzheimer disease. J Neurochem 102:1727-1737; 2007.
Zelcer N, Khanlou N, Clare R, Jiang Q, Reed-Geaghan E G, Landreth G E, Vinters H V, Tontonoz P. Attenuation of neuroinflammation and Alzheimer's disease pathology by liver X receptors. Proc Natl Acad Sci 104:10601-10606; 2007.
Koldamova R, Lefterov I. Role of LXR and ABCA1 in the pathogenesis of Alzheimer's disease implications for a new therapeutic approach. Curr Alzheimer Res 4:171-178; 2007.
Riddell D R, Zhou H, Comery T A, Kouranova E, Lo C F, Warwick H K, Ring R H, Kirksey Y, Aschmies S, Xu J, Kubek K, Hirst W D, Gonzales C, Chen Y, Murphy E, Leonard S, Vasylyev D, Oganesian A, Martone R L, Pangalos M N, Reinhart P H, Jacobsen J S. The LXR agonist TO901317 selectively lowers hippocampal Abeta42 and improves memory in the Tg2576 mouse model of Alzheimer's disease. Mol Cell Neurosci 34:621-628; 2007.
Koldamova R P, Lefterov I M, Staufenbiel M, Wolfe D, Huang 5, Glorioso J C, Walter M, Roth M G, Lazo J S. The liver X receptor ligand TO901317 decreases amyloid beta production in vitro and in a mouse model of Alzheimer's disease. J Biol Chem 280:4079-4088; 2005.
Xiong H, Callaghan D, Jones A, Walker D G, Lue L F, Beach T G, Sue L I, Woulfe J, Xu H, Stanimirovic D B, Zhang W. Cholesterol retention in Alzheimer's brain is responsible for high beta- and gamma-secretase activities and Abeta production. Neurobiol Dis 29:422-437; 2008.

Support for the conclusion that the LXR activators of the present invention can inhibit inflammatory conditions or diseases is provided, e.g., by the following references, which indicate that other LXR activators can activate NFkB, a transcription factor that is the mediator of many inflammatory responses, in a variety of acute and chronic inflammatory diseases:
Joseph S B, Castrillo A, Laffitte B A, Mangelsdorf D J, Tontonoz P. Reciprocal regulation of inflammation and lipid metabolism by liver X receptors. Nature Med 9:213-219; 2003.
Wu S, Yin R, Ernest R, Li Y, Zhelyabovska O, Luo J, Yang Y, Yang Q. Liver X receptors are negative regulators of cardiac hypertrophy via suppressing NF-kappaB signaling. Cardiovasc Res 84:119-126; 2009.
Chang L, Zhang Z, Li W, Dai J, Guan Y, Wang X. Liver-X-receptor activator prevents homocysteine-induced production of IgG antibodies from murine B lymphocytes via the ROS-NF-kappa B pathway. Biochem Biophys Res Commun 357:772-778; 2007.

Support for the conclusion that the LXR activators of the present invention can inhibit or prevent osteoarthritis is provided, e.g., by the following reference, which indicates that other LXR activators exhibit this effect:
Collins-Racie L A, Yang Z, Arai M, Li N, Majumdar M K, Nagpal S, Mounts W M, Domer A J, Morris E, LaVallie E R. Global analysis of nuclear receptor expression and dysregulation in human osteoarthritic articular cartilage: reduced LXR signaling contributes to catabolic metabolism typical of osteoarthritis. Osteoarthritis Cartilage 17:832-842; 2009.

The agents discussed herein can be formulated into various compositions, e.g., pharmaceutical compositions, for use in therapeutic treatment methods. The pharmaceutical compositions can be assembled as a kit. Generally, a pharmaceutical composition of the invention comprises a therapeutically effective amount of a composition of the invention.

A pharmaceutical composition of the invention can comprise a carrier, such as a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. For a discussion of pharmaceutically acceptable carriers and other components of pharmaceutical compositions, see, e.g., Remington's Pharmaceutical Sciences, le ed., Mack Publishing Company, 1990.

A pharmaceutical composition or kit of the invention can contain other pharmaceuticals, in addition to the Hedgehog inhibiting agents of the invention. The other agent(s) can be administered at any suitable time during the treatment of the patient, either concurrently or sequentially.

One skilled in the art will appreciate that the particular formulation will depend, in part, upon the particular agent that is employed, and the chosen route of administration. Accordingly, there is a wide variety of suitable formulations of compositions of the present invention.

Formulations suitable for oral administration can consist of liquid solutions, such as an effective amount of the agent dissolved in diluents, such as water, saline, or fruit juice; capsules, sachets or tablets, each containing a predetermined amount of the active ingredient, as solid, granules or freeze-dried cells; solutions or suspensions in an aqueous liquid; and oil-in-water emulsions or water-in-oil emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers. Suitable formulations for oral delivery can also be incorporated into synthetic and natural polymeric microspheres, or other means to protect the agents of the present invention from degradation within the gastrointestinal tract.

Formulations suitable for parenteral administration (e.g. intravenous) include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The Hedgehog inhibiting LXR agonists of the invention, alone or in combination with other therapeutic agents, can be made into aerosol formulations to be administered via inhalation. These aerosol formulations can be placed into pressurized acceptable propellants, such as dichlorodifluoromethane, propane, nitrogen and the like.

The Hedgehog inhibiting LXR agonists of the invention, alone or in combinations with other therapeutic agents, can be made into suitable formulations for transdermal application and absorption (Wallace et al., 1993, supra). Transdermal electroporation or iontophoresis also can be used to promote and/or control the systemic delivery of the agents and/or pharmaceutical compositions of the present invention through the skin (e.g., see Theiss et al. (1991), Meth. Find. Exp. Chu. Pharmacol. 13, 353-359).

Formulations which are suitable for topical administration include lozenges comprising the active ingredient in a flavor, usually sucrose and acacia or tragacanth; pastilles comprising the active ingredient in an inert base, such as gelatin and glycerin, or sucrose and acacia; mouthwashes comprising the active ingredient in a suitable liquid carrier; or creams, emulsions, suspensions, solutions, gels, creams, pastes, foams, lubricants, sprays, suppositories, or the like.

One skilled in the art will appreciate that a suitable or appropriate formulation can be selected, adapted or developed based upon the particular application at hand.

Dosages for Hedgehog inhibiting LXR agonists of the invention can be in unit dosage form, such as a tablet or capsule. The term “unit dosage form” as used herein refers to physically discrete units suitable as unitary dosages for animal (e.g. human) subjects, each unit containing a predetermined quantity of an agent of the invention, alone or in combination with other therapeutic agents, calculated in an amount sufficient to produce the desired effect in association with a pharmaceutically acceptable diluent, carrier, or vehicle.

One skilled in the art can easily determine the appropriate dose, schedule, and method of administration for the exact formulation of the composition being used, in order to achieve the desired effective amount or effective concentration of the agent in the individual patient. One skilled in the art also can readily determine and use an appropriate indicator of the “effective concentration” of the compounds of the present invention by a direct or indirect analysis of appropriate patient samples (e.g., blood and/or tissues). Assays of Hedgehog inhibition can calibrate dosage for particular LXR agonists.

The dose of a Hedgehog inhibiting LXR agonist of the invention, or composition thereof, administered to an animal, particularly a human, in the context of the present invention should be sufficient to elicit at least a therapeutic response in the individual over a reasonable time frame. The dose used to achieve a desired concentration in vivo will be determined by the potency of the particular Hedgehog inhibiting LXR agonist employed, the pharmacodynamics associated with the agent in the host, the severity of the disease state of infected individuals, as well as, in the case of systemic administration, the body weight and age of the individual. The size of the dose also will be determined by the existence of any adverse side effects that may accompany the particular agent, or composition thereof, employed. It is generally desirable, whenever possible, to keep adverse side effects to a minimum.

For example, a dose can be administered in the range of from about 5 ng (nanograms) to about 1000 mg (milligrams), or from about 100 ng to about 600 mg, or from about 1 mg to about 500 mg, or from about 20 mg to about 400 mg. For example, the dose can be selected to achieve a dose to body weight ratio of from about 0.0001 mg/kg to about 1500 mg/kg, or from about 1 mg/kg to about 1000 mg/kg, or from about 5 mg/kg to about 150 mg/kg, or from about 20 mg/kg to about 100 mg/kg. For example, a dosage unit can be in the range of from about 1 ng to about 5000 mg, or from about 5 ng to about 1000 mg, or from about or from about 100 ng to about 600 rug, or from about 1 mg to about 500 mg, or from about 20 mg to about 400 mg, or from about 40 mg to about 200 mg of a compound of according to the present invention. A dose can be administered once per day, twice per day, four times per day, or more than four times per day as required to elicit a desired therapeutic effect. For example, a dose administration regimen can be selected to achieve a blood serum concentration of a compound of the present invention in the range of from about 0.01 to about 20000 nM, or from about 0.1 to about 15000 nM, or from about 1 to about 10000 nM, or from about 20 to about 10000 nM, or from about 100 to about 10000 nM, or from about 200 to about 5000 nM, or from about 1000 to about 5000 nM. For example, a dose administration regime can be selected to achieve an average blood serum concentration with a half maximum dose of a compound of the present invention in the range of from about 1 μg/L (microgram per liter) to about 2000 μg/L, or from about 2 μg/L to about 1000 μg/L, or from about 5 μg/L to about 500 μg/L, or from about 10 μg/L to about 400 μg/L, or from about 20 μg/L to, about 200 μg/L, or from about 40 μg/L to about 100 μg/L.

A therapeutically effective dose of a Hedgehog inhibiting LXR agonist or other agent useful in this invention is one which has a positive clinical effect on a patient, e.g. as measured by the ability of the agent to reduce cell proliferation. The therapeutically effective dose of each agent can be modulated to achieve the desired clinical effect, while minimizing negative side effects. The dosage of the agent may be selected for an individual patient depending upon the route of administration, severity of the disease, age and weight of the patient, other medications the patient is taking and other factors normally considered by an attending physician, when determining an individual regimen and dose level appropriate for a particular patient.

When given in combined therapy, the other agent can be given at the same time as the Hedgehog inhibiting LXR agonist, or the dosing can be staggered as desired. The two (or more) drugs also can be combined in a composition. Doses of each can be less when used in combination than when either is used alone.

The invention may include treatment with an additional agent which acts independently or synergistically with the Hedgehog inhibitor. Additional classes of agents which may be useful in this invention alone or in combination with Hedgehog inhibiting LXR agonists include, but are not limited to known anti-proliferative agents. Those skilled in the art would be able to determine the accepted dosages for each of the therapies using standard therapeutic dosage parameters.

The invention may include a method of systemic delivery or localized treatment alone or in combination with administration of other agent(s) to the patient.

Another embodiment of the invention is a kit useful for any of the methods disclosed herein, either in vitro or in vivo. Such a kit can comprise one or more of the Hedgehog inhibiting LXR agonists or pharmaceutical compositions discussed herein. Optionally, the kits comprise instructions for performing the method. Optional elements of a kit of the invention include suitable buffers, pharmaceutically acceptable carriers, or the like, containers, or packaging materials. The reagents of the kit may be in containers in which the reagents are stable, e.g., in lyophilized form or stabilized liquids. The reagents may also be in single use form, e.g., in single dosage form. A skilled worker will recognize components of kits suitable for carrying out any of the methods of the invention.

In the foregoing and in the following examples, all temperatures are set forth in uncorrected degrees Celsius; and, unless otherwise indicated, all parts and percentages are by weight.

### Examples

When a “statistically significant amount” is referred to in the following Examples, this depends on a number of factors, such as the technique of the experimenter and the quality of the equipment used. For example, in certain cases, a statistically significant amount may be a change of 1%. In other cases, a statistically significant amount can be represented by a change of at least about 5%, 10%, 20%, 50%, 75%, double, or more. In relation to inhibition, the significant reduction may be to a level of less than about 90%, 75%, 50%, 25%, 10%, 5%, 1%, or less.

### 1) Structures and Names of Oxysterol Molecules Described Herein: (e.g., Formula I, Formula II, Oxy16, Oxy22, Oxy30, Oxy31, Oxy35, Oxy37, Oxy43, Oxy44, Oxy45, Oxy47)

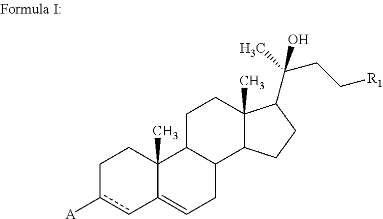

wherein A is hydrogen or hydroxy,

wherein [symbol] is a single or a double bond,

wherein R₁ is selected from the group consisting of

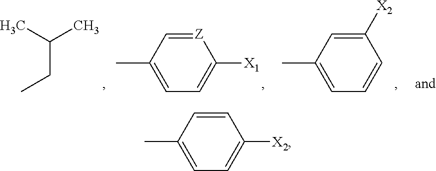

wherein Z is nitrogen that can be anywhere in the ring,

wherein X₁ can be bonded to any position on the ring, and is selected from the group consisting of hydrogen, fluorine, chlorine, bromine, and iodine, and

wherein X₂ is selected from the group consisting of fluorine, chlorine, bromine, and iodine.

In embodiments of the invention R₁ is selected from the group consisting of

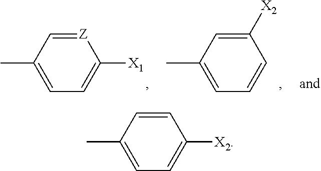

### Or R₁ is

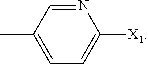

Or X₁ is selected from the group consisting of hydrogen, fluorine, and chlorine and

X₂ is selected from the group consisting of fluorine and chlorine.

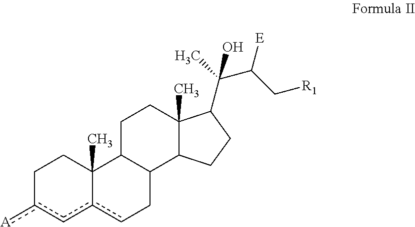

wherein A is selected from the group consisting of hydrogen, hydroxy, or oxygen,

wherein [symbol] is a single or a double bond,

wherein E is hydrogen or hydroxy,

wherein R₁ is selected from the group consisting of

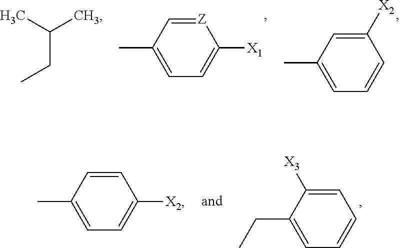

wherein Z is nitrogen that can be anywhere in the ring,

wherein X₁ can be bonded to any position on the ring and is selected from the group consisting of hydrogen, fluorine, chlorine, bromine, and iodine, and

wherein X₂ is selected from the group consisting of fluorine, chlorine, bromine, and iodine,

wherein X₃ can be bonded to any position on the ring and is selected from the group consisting of hydrogen, fluorine, chlorine, bromine, and iodine.

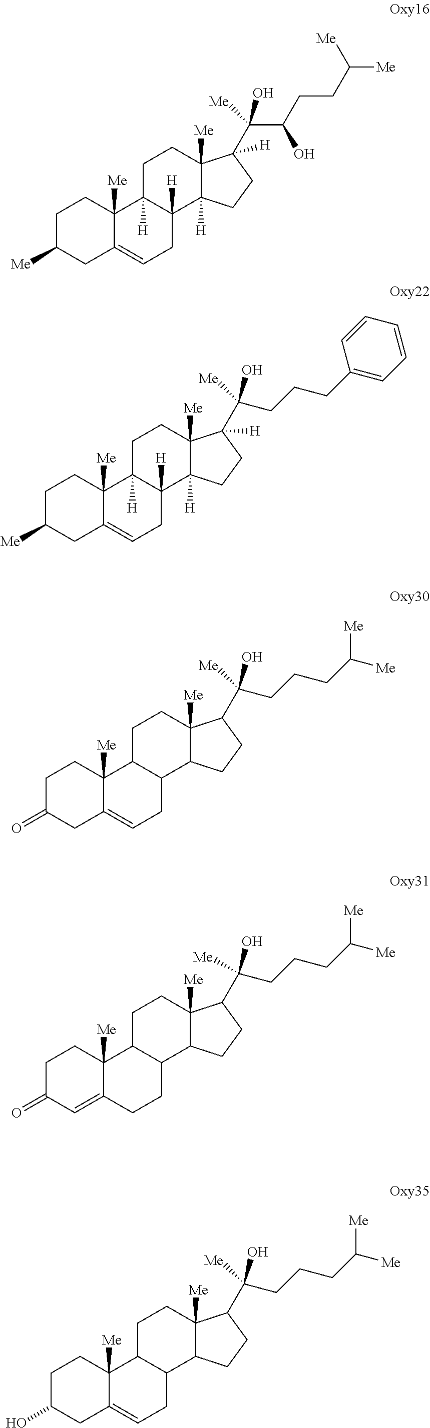

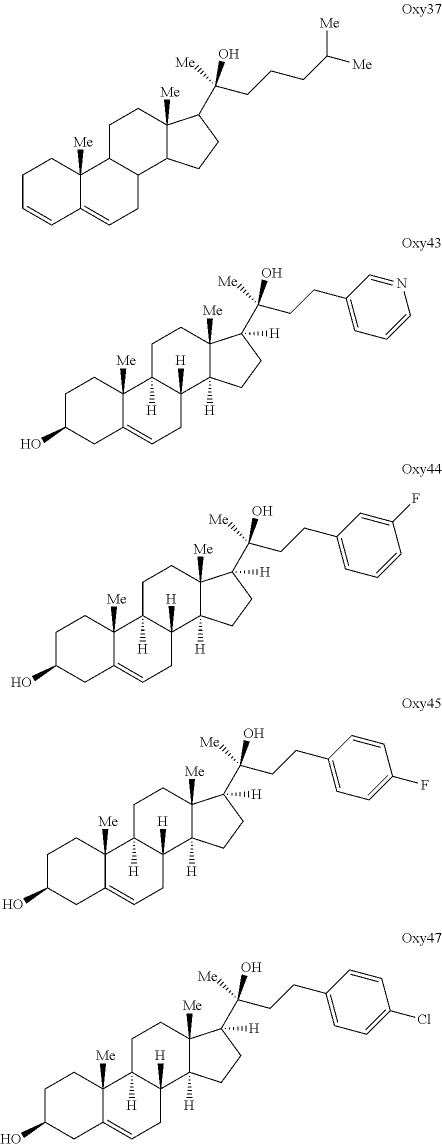

### 2) The Oxysterol Molecules, Oxy16, Oxy22, Oxy30, Oxy 31, Oxy35, Oxy37, Oxy43, Oxy44, Oxy45, Oxy47, Activate LXR Signaling in Bone Marrow Stromal Cells

This was measured by the ability of these molecules to induce the expression of LXR-target genes, including ABCA1, in M2-10B4 bone marrow stromal cells (MSC) after 48 hours of treatment (Table 1). As the inventors previously reported, activation of LXR can result in the inhibition of Hedgehog signaling in various cell types. Since aberrant Hedgehog signaling in cancer cells has been reported to be a cause of tumor formation, it is suggested (without wishing to be bound by any particular mechanism), that the inhibitory effects of LXR activating oxysterols on tumor cells may be due, at least in part, to inhibition of Hedgehog signaling.

TABLE 1
Effect of small molecule oxysterols on ABCA1 gene expression. RNA
from M2-10B4 cells treated with 2 μM of each oxysterol for 48 hours
was analyzed by Q-RT-PCR for the expression of LXR target
gene ABCA1 and the house keeping gene GAPDH for normalization.
Data are reported as fold induction relative to untreated control cells.

| Treatment | Fold Induction ± SD |
|---|---|
| Oxy16 | 2.1 ± 0.5 |
| Oxy22 | 2.2 ± 0.4 |
| Oxy30 | 2.8 ± 0.8 |
| Oxy31 | 2.0 ± 0.3 |
| Oxy35 | 4.0 ± 1.2 |
| Oxy37 | 2.0 ± 0.1 |
| Oxy43 | 2.5 ± 0.8 |
| Oxy44 | 2.5 ± 0.5 |
| Oxy45 | 3.5 ± 0.5 |
| Oxy47 | 1.8 ± 0.5 |
| Oxy17 | 1.0 ± 0.1 |

### 3) LXR Activation by Oxysterols of the Invention and by the Pharmacologic LXR Ligand TO901317 (TO) Inhibits Clonogenic Growth of Human Pancreatic Cancer Cells

The human pancreatic cancer cell line L3.6p1 was seeded into 6 well plates in Advanced RPMI1640 media containing 1% fetal bovine serum and treated with an ethanol vehicle control or the commercially available oxysterol 22R-hydroxycholesterol (22R) (a positive control that is known to activate LXR), or synthetic oxysterols Oxy17 (which does not activate LXR), Oxy16, Oxy30, or T0901317 LXR ligand for 72 hours (all at 5 or 10 μM). Following treatment cells were harvested by washing cells twice with phosphate buffered saline (PBS) followed by enzymatically detaching with trypsin/EDTA. Cells were collected then washed twice with PBS. Cells were counted then resuspended in 500 uL of media. The volume of cells required for 2,000 cells from the control group was removed from each group then mixed with methylcellulose (1.2%) containing 30% fetal bovine serum, 1% bovine serum albumin, 10-4 M 2-mercaptoethanol, and 2 mM L-glutamine. Cells were plated in low-attachment 6 well plates (1 ml/well), each group being plated in triplicate. Following 10 days of incubation, tumor cell colonies consisting of >40 cells were counted using an inverted microscope. Results are presented as the percentage of colonies from each treatment group compared to the control group (Table 2).

TABLE 2
Effect of LXR activation on clonogenic growth of
L3.6 human pancreatic cancer cells.

| | Dose | Raw | Normalized Colony |
|---|---|---|---|
| Treatment | (μM) | Colony # | # (% of control) |
| Control | — | 243 | 100 |
| 22R | 5 | 153 | 63 |
| 22R | 10 | 142 | 59 |
| Oxy17 | 5 | 224 | 92 |
| Oxy17 | 10 | 230 | 95 |
| Oxy16 | 5 | 53 | 22 |
| Oxy16 | 10 | 24 | 10 |
| Oxy30 | 5 | 96 | 39 |
| Oxy30 | 10 | 100 | 41 |
| TO | 5 | 100 | 41 |
| TO | 10 | 37 | 15 |

### 4) LXR Activation by Oxysterols of the Invention and by the Pharmacologic LXR Ligand TO901317 (TO) Inhibits Clonogenic Growth of Human Acute Lymphocytic Leukemia (ALL) Cells

A similar experiment to that shown above for pancreatic cancer cells was performed using the human ALL cells, REH (Table 3).

TABLE 3
Effect of LXR activation on clonogenic
growth of REH human ALL cells.

| | Dose | Raw | Normalized Colony |
|---|---|---|---|
| Treatment | (μM) | Colony # | # (% of control) |
| Control | — | 92 | 100 |
| 22R | 0.1 | 33 | 36 |
| 22R | 0.5 | 2 | 2 |
| Oxy17 | 0.1 | 87 | 95 |
| Oxy17 | 0.5 | 72 | 78 |
| Oxy16 | 0.1 | 65 | 71 |
| Oxy16 | 0.5 | 14 | 15 |
| Oxy45 | 0.1 | 40 | 43 |
| Oxy45 | 0.5 | 0.5 | 0.5 |
| TO | 0.1 | 44 | 48 |
| TO | 0.5 | 34 | 37 |

### 5. Method of Synthesis for Oxysterols of the Invention

### (2R,3R)-2-((3S,8S,9S,10R,13S,14S,17S)-3-Hydroxy-10,13-dimethyl-2,3,4,7,8,9,10,11, 12,13,14,15,16,17-tetradecahydro-1H-cyclopenta[a]phenanthren-17-yl)-6-methyl-heptane-2,3-diol, Oxy16

The stereoselective synthesis of Oxy16 was carried out according to published procedures (44). The silylated pregnenolone was subjected to stereoselective addition of the anion of 4-methyl-1-pentyne formed by reaction of the acetylene with n-butyllithium to provide the propargylic alcohol in 84% yield followed by hydrogenation in the presence of Lindlar catalyst give a mixture of the (Z)- and (E)-allylic alcohols (90:10). Both isomers were separated chromatographically to afford the (Z)-isomer in 68% yield and the (E)-isomer in 7% yield. Regioselective epoxidation of the (Z)-allylic alcohol under VO(acac)₂/tert-butyl hydroperoxide (TBHP) conditions pro-vided a 1:1 mixture of the diastereomeric epoxides. These were separated using silica gel column chromatography to give the pure β- and α-epoxide in 39% and 49% yield, res-pectively. The regioselective ring opening of the α-epoxide with LiAlH₄ gave the (20R,22R) diol in 80% yield. Deprotection of the silyl ethers with tetrabutylammonium fluoride (TBAF) afforded the desired triol Oxy16 in quantitative yield, the spectroscopic data of which was identical to those reported in the literature.¹

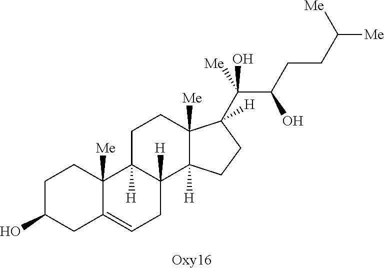

### 1-((3S,8S,9S,10R,13S,14S,17S)-2,3,4,7,8,9,10,11,12,13,14,15,16,17-Tetradecahydro-3-[(1,1-dimethylethyl)dimethylsilyloxy]-10,13-dimethyl-1H-cyclopenta[a]phenanthren-17-yl)ethanone, 2

To a stirred solution of pregnenolone (5.0 g, 15.8 mmol) in anhyd-rous dimethylformamide (DMF, 180 mL) was added imidazole (2.7 g. 39.7 mmol). The reaction was allowed to stir for 20 min followed by slow addition of tert-butyldimethyl-silyl chloride (3.6 g, 23.9 mmol). After stirring for 12 h at ambient temperature, the reaction mixture was poured over ice. The precipitates were collected and dissolved in diethyl ether. The organic phases were washed with brine, dried over Na₂SO₄ and evaporated in vacuo to yield compound 2 (6.7 g, 15.6 mmol, 98%) as a white powder which was used without further purification. The spectroscopic data was identical to those reported in the literature (45)

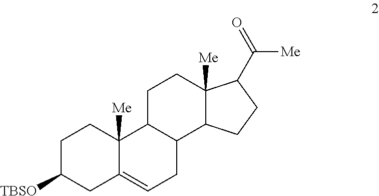 [SMILES: CC(=O)C1CCC2C3CC=C4C[C@@H](O[Si](C)(C)C(C)(C)C)CC[C@]4(C)C3CC[C@]12C]

### I-2-((3S,8S,9S,10R,13S,14S,17S)-3-(tert-Butyldimethylsilyloxy)-10,13-dimethyl-2, 3,4,7,8,9,10,11,12,13,14,15,16,17-tetradecahydro4H-cyclopenta[a]phenanthren-17-yl)but-3-yn-2-ol, 3

To a solution of trimethylsilylacetylene (500 mg, 5.01 mmol) in 5.0 mL of anhydrous THF, was added n-butyllithium (1.0 mL, 2.5 mmol) at 0° C. After 30 min, a solution of 2 (500 mg, 1.58 mmol) in THF (10 mL) was slowly added. The reaction was quenched after 1 h with satd. NH₄Cl and extracted twice with diethyl ether. The organic layers were combined and washed with satd. NaCl, dried over Na₂SO₄ and evapo-rated in vacuo to afford a crude solid, which upon treatment with potassium carbonate (600 mg, 4.34 mmol) in 6.0 mL mixture of methanol/tetrahydrofuran (5:1 v/v) yielded the crude desilylated propargyl alcohol. The solvent was removed and the residue was extracted with diethyl ether. The organic phases were collected, dried over Na₂SO₄ and evaporated in vacuo followed by column chromatography on silica gel using hexane-diethyl ether (2:1 v/v) to afford 3 (360 mg, 78% over 2 steps) as a white solid.

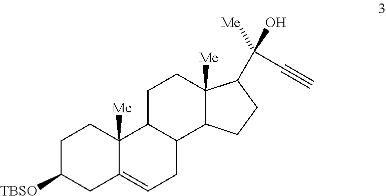 [SMILES: C#C[C@](C)(O)C1CCC2C3CC=C4C[C@@H](O[Si](C)(C)C(C)(C)C)CC[C@]4(C)C3CC[C@@]21C]

¹H NMR (400 MHz, CDCl₃) δ: 5.32-5.31 (1H, m), 3.52-3.44 (1H, m), 2.51 (1H, s), 2.23-2.12 (5H, m), 1.99-1.95 (2H, m), 1.82-1.57 (9H, m), 1.49 (3H, s), 1.28-1.04 (5H, m), 0.98 (3H, s), 0.96 (3H, s), 0.83 (9H, s), 0.06 (6H, s). ¹³C NMR (CDCl₃, 100 MHz) δ: 141.7, 121.0, 87.5, 73.8, 72.6, 71.3, 60.0; 55.3, 50.1, 43.3, 42.8, 40.3, 37.4, 36.6, 32.8, 32.1, 31.9, 31.4, 26.0, 25.1, 24.2, 20.8, 19.5, 18.3, 13.4, −4.6.

### (3S,8S,9S,10R,13S,14S,17R)-17-(2,3,4,7,8,9,10,11,12,13,14,15,16,17-Tetradecahydro-17-((S)-2-Hydroxy-5-phenylpent-2-yl)10,13-dimethyl-1H-cyclopenta[a]phenanthren-3-ol, Oxy22

To a stirred suspension of magnesium turnings (106.7 mg, 4.4 mmol) in anhydrous diethyl ether (3.5 mL) was added (3-bromopropyl)benzene (199.0 mg, 1.22 mmol). After stirring under reflux for 2 h, the initially produced Grignard reagent was cannulated into a solution of the protected pregnenolone 2 (300 mg, 0.70 mmol) in anhydrous THF (20 mL) and left under reflux for an additional 2 h. The mixture was cooled in an ice bath and treated with satd. NH₄Cl. The solution was filtered through Celite and the precipitate washed three times with diethyl ether. The filtrate was extracted twice with diethyl ether. The organic layers were combined and washed with satd. NaCl, dried over Na₂SO₄ and evaporated in vacuo to afford a residue, which was subjected to column chromatography on silica gel. Elution with hexane-diethyl ether (2:1 v/v) afforded the alcohol followed by desilylation with a 1.0 M solution of tetrabutylammonium fluoride in THF (1.0 mL, 1.0 mmol), and was allowed to stir at 20° C. After stirring for 12 h, the reaction was treated with water and extracted three times with diethyl ether and the organic layer was washed with satd. NaCl. The organic phases were collected, dried over Na₂SO₄ and concentrated in vacuo to give an oil. Flash column chromatography of this oil (silica gel, 1:2 hexane/diethyl ether) yielded Oxy22 (170.0 mg, 56% over 2 steps) as a white powder.

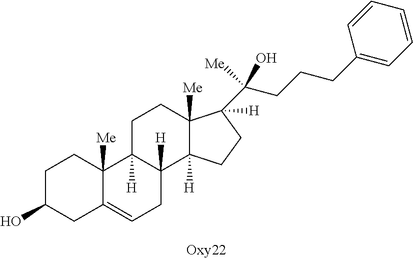

¹H NMR (CDCl₃; 400 MHz) δ: 7.30-7.26 (2H, m), 7.20-7.19 (3H, m), 5.35 (1H, m), 3.56-3.48 (1H, m), 2.61-2.56 (2H, m), 2.28-2.23 (2H, m), 2.20-2.17 (1H, m), 2.08-2.05 (1H, m), 1.85-1.39 (16H, m), 1.26 (3H, s), 1.18-1.07 (4H, m), 1.00 (3H, s), 0.85 (3H, s). ¹³C NMR (CDCl₃, 100 MHz) δ: 142.5, 140.8, 128.4, 128.3, 125.8, 121.6, 75.2, 71.7, 57.6, 56.9, 50.0, 43.6, 42.7, 42.3, 40.1, 37.2, 36.5, 31.8, 31.6, 31.3, 26.4, 26.41, 23.8, 22.3, 20.9, 19.4, 13.6.

### General Method for the Preparation of Oxy43-47

### (3S,8S,9S,10R,13S,14S,17S)-17-((S)-2-Hydroxy-4-(yridine-3-yl)butan-2-yl)-10,13-dimethyl-2,3,4,7,8,940,11,12,13,14,15,16,17-tetradecahydro-1H-cyclopenta-[a]-phenanthren-3-ol, Oxy43

To a solution of the propargyl alcohol 3 (300 mg, 0.66 mmol) in anhydrous tetrahydrofuran (THF, 5.0 mL) was added diisopropylamine (5.0 mL), 3-bromopyridine (400 mg, 2.5 mmol), Pd(PPh₃)₄ (42 mg, 0.036 mmol) and CuI (16 mg, 0.84 mmol) (46). The reaction mixture was left under reflux over N₂ atmosphere for 12 h. The solvent was removed under reduced pressure followed by flash column chromato-graphy (silica gel, 1:1 diethyl ether/hexane v/v) to afford the aryl acetylene product (150 mg, 43%) as an off-white powder. Catalytic hydrogenation over Pd/C (10% mol) in 1:1 dichloromethane:95% EtOH (3.0 mL) under a H₂ atmosphere was carried out for 12 h, the crude mixture was filtered through Celite using ethyl acetate and the solvent was removed under reduced pressure. The mixture was then treated with a 1.0 M solution of TBAF in THF (2.0 mL, 2.0 mmol) and it was allowed to stir at 20° C. for 12 h. The reaction was treated with water and extracted three times with diethyl ether and the organic layer was washed with satd. NaCl. The organic phases were collected, dried over Na₂SO₄ and concentrated in vacuo to give an oil. Flash column chromatography of this oil (silica gel, 1:3 hexane/diethyl ether v/v) afforded Oxy43 in quantitative yield as a white powder.

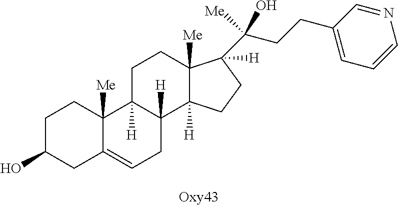

¹H NMR (CDCl₃; 400 MHz) δ: 8.39 (2H, m), 7.51 (1H, d, J=6.4 Hz), 7.31 (1H, m), 5.36-5.35 (1H, m), 3.53-3.45 (1H, m), 2.65-2.63 (2H, m), 2.29-1.49 (20H, m), 1.38 (3H, s), 1.25-1.04 (4H, m), 1.01 (3H, s), 0.88 (3H, s). ¹³C NMR (CDCl₃, 100 MHz) δ: 150.0, 147.2, 140.8, 138.4, 135.8, 123.7, 121.5, 75.0, 71.7, 58.2, 56.9, 50.0, 45.1, 42.8, 42.3, 40.2, 37.3, 36.5, 31.8, 31.6, 31.3, 27.7, 26.2, 23.8, 22.5, 20.9, 19.4, 13.7.

### (3S,8S,9S,10R,13S,14S,17S)-17-((S)-4-(4-Fluorophenyl)-2-hydroxybutan-2-yl)-10,13-dimethyl-2,3,4,7,8,9,10,11,12,13,14,15,16,17-tetradecahydro-1H-cyclopenta-[a]-phenanthren-3-ol, Oxy44

Prepared by the same method as for Oxy43, using 3-fluoro-1-bromobenzene (404 mg, 2.3 mmol). Purification of the crude material via column chromatography on silica gel using diethyl ether-hexane (1:3 v/v) afforded the aryl acetylene product (139 mg, 38%) as an off-white powder. Catalytic hydrogenation with Pd/C (10% mol) in ethyl acetate (3.0 mL) under a H₂ atmosphere for 12 h followed by desilylation with a 1.0 M solution of TBAF afforded Oxy44 as a white solid in quantitative yield.

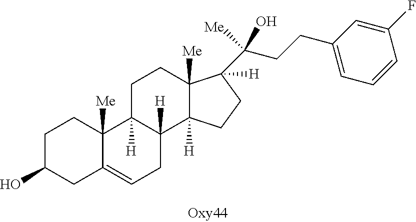

¹H NMR (CDCl₃; 400 MHz) δ: 7.25-7.19 (1H, m), 6.96-6.84 (3H, m), 5.36-5.35 (1H, m), 3.56-3.50 (1H, m), 2.28-1.48 (21H, m), 1.36 (3H, s), 1.25-1.03 (5H, m), 1.01 (3H, s), 0.88 (3H, s). ¹³C NMR (CDCl₃, 100 MHz) δ: 164.1, 161.7, 145.4, 145.3, 140.8, 129.8, 129.7, 123.98, 123.95, 121.6, 115.3, 115.0, 112.7, 112.5, 75.0, 71.8, 58.1, 56.9, 50.0, 45.2, 42.8, 42.3, 40.2, 37.2, 36.5, 31.8, 31.6, 31.3, 30.4, 26.2, 23.8, 22.5, 20.9, 19.4, 13.7. ¹⁹F (CDCl₃; 400 MHz) δ: −114.4.

### (3S,8S,9S,10R,13S,14S,17S)-17-((S)-4-(4-Fluorophenyl)-2-hydroxybutan-2-yl)-10,13-dimethyl-2,3,4,7,8,9,10,11,12,13,14,15,16,17-tetradecahydro-1H-cyclopenta-M-phenanthren-3-ol, Oxy45

Prepared by the same method as for Oxy43, using 4-fluoro-1-bromobenzene (404 mg, 2.3 mmol). Purification of the crude material via column chromatography on silica gel using diethyl ether-hexane (1:3 v/v) afforded the aryl acetylene product (232 mg, 63%) as an off-white powder. Catalytic hydrogenation with Pd/C (10% mol) in ethyl acetate (3.0 mL) under a H₂ atmosphere for 12 h followed by desilylation with a 1.0 M solution of TBAF afforded Oxy45 as a white solid in quantita-tive yield.

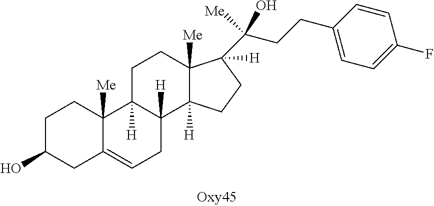

¹H NMR (CDCl₃; 400 MHz) δ: 7.17-7.10 (2H, m), 6.97-6.92 (2H, m), 5.35-5.34 (1H, m), 3.54-3.47 (1H, m), 2.61-2.58 (2H, m), 2.28-1.49 (19H, m), 1.36 (3H, s), 1.25-1.20 (5H, m), 1.01 (3H, s), 0.88 (3H, s). ¹³C NMR (CDCl₃, 100 MHz) δ: 162.4, 160.0, 140.8, 138.3, 138.2, 129.64, 129.56, 121.6, 115.2, 115.0, 75.1, 71.2, 58.1, 56.9, 50.0, 45.7, 42.7, 42.3, 40.2, 37.3, 36.5, 31.8, 31.6, 31.3, 29.8, 26.3, 23.8, 22.5, 20.9, 19.4, 13.7. ¹⁹F (CDCl₃; 400 MHz) δ: −118.5.

### (3S,8S,9S,10R,13S,14S,17S)-17-((S)-4-(4-Chlorophenyl)-2-hydroxybutan-2-yl)-10,13-dimethyl-2,3,4,7,8,9,10,11,12,13,14,15,16,17-tetradecahydro-1H-cyclopenta-[a]-phenanthren-3-ol, Oxy47

Prepared by the same method as for Oxy43, using 1-chloro-4-iodobenzene (500 mg, 2.1 mmol). Purification of the crude material via column chromatography on silica gel using diethyl ether-hexane (1:3 v/v) afforded the aryl acetylene product (260 mg, 69%) as an off-white powder. Catalytic hydrogenation with Pd/C (10% mol) in ethyl acetate (3.0 mL) under a H₂ atmosphere for 12 h followed by desilylation with a 1.0 M solution of TBAF afforded Oxy47 as a white solid in quantitative yield.

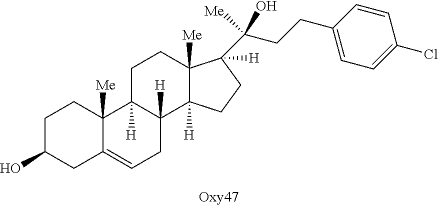

¹H NMR (CDCl₃; 400 MHz) δ: 7.23 (2H, d, J=6.6 Hz), 7.10 (2H, d, J=6.6 Hz), 5.35-5.34 (1H, m), 3.52-3.48 (1H, m), 2.60-2.58 (2H, m), 2.30-1.44 (20H, m), 1.35 (3H, s), 1.26-1.04 (4H, m), 1.00 (3H, s), 0.87 (3H, s). ¹³C NMR (CDCl₃, 100 MHz) δ: 141.2, 140.8, 131.4, 129.7, 128.5, 121.5, 75.1, 71.7, 58.1, 56.9, 50.0, 45.5, 42.7, 42.3, 40.2, 37.3, 36.5, 31.8, 31.6, 31.3, 30.0, 26.2, 23.8, 22.5, 20.9, 19.4, 13.7.

### (10R,13S)-17-((S)-2-Hydroxy-6-methylheptan-2-yl)-10,13-dimethyl-4,7,8,9,10,11,12, 13,14,15,16,17-dodecahydro-1H-cyclopenta[a]phenanthren-3(2H)-one, Oxy30, and (10R,13S)-17-((S)-2-Hydroxy-6-methylheptan-2-yl)-10,13-dimethyl-6,7,8,9,10,11,12, 13,14,15,16,17-dodecahydro-1H-cyclopenta[a]phenanthren-3(2H)-one, Oxy31

To a stirred solution of 20S-cholesterol (19.0 mg, 0.047 mmol) and 4 Å molecular sieves in dichloromethane (5 mL) was added N-methylmorpholine N-oxide (NMO, 6.6 mg, 0.057 mmol) followed by tetrapropylammonium perruthenate (TPAP, 1.7 mg, 0.005 mmol) at 23° C. After 1 h, the reaction mixture was passed through Celite, and the filtrate was concentrated. Purification by flash column chromatography (20% ethyl acetate in hexane) yielded Oxy30 (6.0 mg, 32%) and Oxy31 (4.0 mg, 21%). Oxy30 ¹H NMR (400 MHz, CDCl₃): δ 5.35 (1H, m), 3.28 (1H, dd, J=16.5, 2.7 Hz), 2.82 (1H, dd, J=16.5, 2.0 Hz), 2.54-0.81 (25H, m), 1.28 (3H, s), 1.19 (3H, s), 0.90 (3H, s), 0.87 (6H, d, J=6.6 Hz). Oxy31 ¹H NMR (400 MHz, CDCl₃): δ 6.18 (1H, d, J=0.7 Hz), 2.75-0.83 (27H, m), 1.29 (3H, s), 1.17 (3H, s), 0.91 (3H, s), 0.88 (6H, d, J=6.6 Hz).

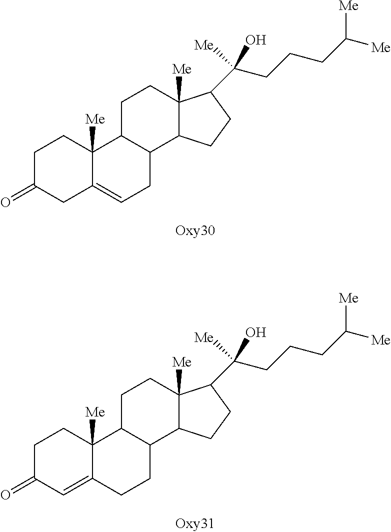 [SMILES: CC(C)CCC[C@](C)(O)C1CCC2C3CC=C4CC(=O)CC[C@]4(C)C3CC[C@@]21C ; CC(C)CCC[C@](C)(O)C1CCC2C3CCC4=CC(=O)CC[C@]4(C)C3CC[C@@]21C]

### (3R,10R,13S)-17-((S)-2-Hydroxy-6-methylheptan-2-yl)-10,13-dimethyl-2,3,4,7,8,9, 10,11,12,13,14,15,16,17-tetradecahydro-1H-cyclopenta[a]phenanthren-3-ol (3-epi-20S-cholesterol), Oxy35

To a stirred solution of 20S-cholesterol (70 mg, 0.19 mmol) and 4 Å molecular sieves in dichloromethane (10 mL) was added NMO (31 mg, 0.26 mmol) followed by TPAP (6 mg, 0.02 mmol) at 0° C. After 1 h, the reaction mixture was passed through Celite, and the filtrate was concentrated. Purification by flash column chromatography (20% ethyl acetate in hexane) yielded Oxy30 (35 mg, 50%). ¹H NMR δ 5.35 (m, 1H), 3.28 (dd, 1H, J=16.5, 2.7 Hz), 2.82 (dd, 1H, J=16.5, 2.0 Hz), 2.54-0.81 (m, 25H), 1.28 (s, 3H), 1.19 (s, 3H), 0.90 (s, 3H), 0.87 (d, 6H, J=6.6 Hz). To a 1.0 M solution of L-selectride in THF (0.22 mL, 0.22 mmol) was added a solution of Oxy30 (34 mg, 0.09 mmol) in THF (1 mL) at −78° C. After 2 h, the reaction was quenched with satd. NH₄Cl (5 mL) and the crude was isolated by ethyl acetate extraction. Concentration gave an oily product which was purified by flash column chromatography. Elution with 33% ethyl acetate in hexane gave Oxy 35 (26 mg, 75%) as a white solid. ¹H NMR (400 MHz, CDCl₃): δ 5.41 (1H, m), 4.15 (1H, br s), 4.02 (1H, m), 2.63-0.84 (27H, m), 1.28 (3H, s), 1.01 (3H, s), 0.87 (6H, d, J=6.3 Hz), 0.87 (s, 3H).

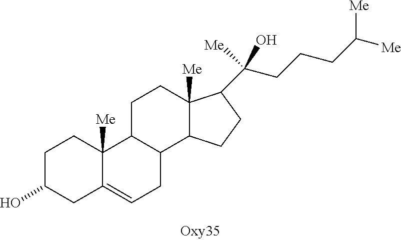 [SMILES: CC(C)CCC[C@](C)(O)C1CCC2C3CC=C4C[C@H](O)CC[C@]4(C)C3CC[C@@]21C]

### (2S)-2-((10R,13S)-10,13-Dimethyl-2,7,8,9,10,11,12,13,14,15,16,17-dodecahydro-1H-cyclopenta[a]phenanthren-17-yl)-6-methylheptan-2-ol, Oxy37

To a solution of Oxy 35 (15.0 mg, 0.037 mL), pyridine (0.015 mL, 0.186 mmol) in dichloromethane (3 mL) was added a solution of methanesulfonyl chloride in dichloromethane (0.007 mL, 0.093 mmol) at 0° C. The reaction was allowed to warm to 23° C. and stirred overnight. The reaction was quenched with 50% NH₄Cl (5 mL) and extracted with ethyl acetate (10 mL). The combined organic layers were dried over MgSO₄, concentrated under vacuum and purified column chromatography (33% ethyl acetate in hexane) to yield 14.9 mg (83%) of the 3α-methanesulfonate. This sulfonate (13.0 mg, 0.027 mmol) was dissolved in DMF (3 mL). Sodium azide (8.8 mg, 0.135 mmol) was added to the mixture and the reaction mixture was heated to 50° C. After cooling to room temperature, the reaction was quenched with 50% NH₄Cl (10 mL) and extracted with ethyl acetate (10 mL). The combined organic layers were dried over MgSO₄, concentrated under vacuum and purified column chromatography (20% ethyl acetate in hexane) to yield 1.8 mg (18%) of the 3β-azido compound and 3.6 mg (35%) of Oxy37. ¹H NMR (400 MHz, CDCl₃): δ 5.93 (1H, m), 5.60 (1H, m), 5.39 (1H, m), 2.21-0.80 (25H, m), 1.28 (3H, s), 0.96 (3H, s), 0.89 (3H, s), 0.87 (6H, d, J=6.6 Hz).

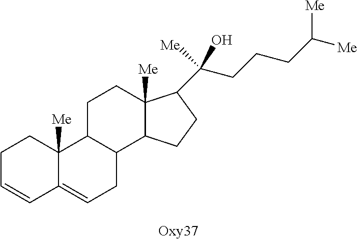 [SMILES: CC(C)CCC[C@](C)(O)C1CCC2C3CC=C4C=CCC[C@]4(C)C3CC[C@@]21C]

### 6. Additional Data

The following data provide further support for the inhibitory effects of liver X receptor (LXR) ligands and LXR activating oxysterols for the inhibition of Hedgehog (Hh) signaling and clonogenic growth of human cancer cells. Human osteosarcoma cells Saos-2 and U2OS were used as a model for studying human solid bone tumors.

A. Saos-2 Osteosarcoma Cells Express LXRα and LXRβ rRNA

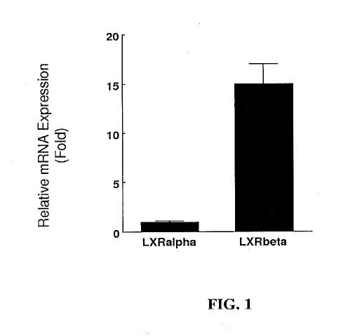

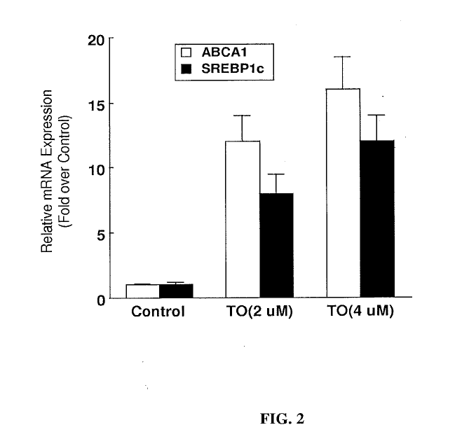

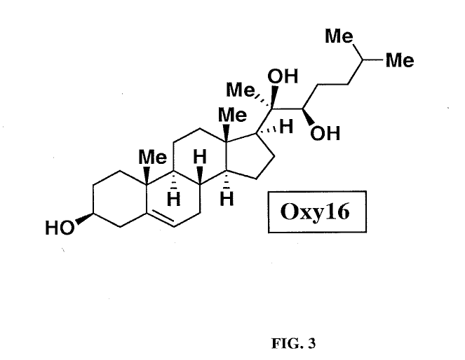

We found that in confluent cultures of Saos-2 cells both LXRβ and LXRβ are expressed, with greater expression of LXR than LXRα (FIG. 1). Culturing the cells in varying serum (FBS) concentrations from 1% to 10% had no effect on LXRs or LXR target gene expression levels at baseline. Furthermore, treatment of Saos-2 cells with TO caused the robust expression of LXR target genes ABCA1 and SREBP1c (FIG. 2). In addition, Saos-2 treatment with specific naturally occurring oxysterols including 22I-hydroxycholesterol (22R) and 20(S)-hydroxycholesterol (20S) that are known physiological ligands of LXR as well as a synthetic oxysterol activator of LXR developed in our laboratory, Oxy16 (FIG. 3), resulted in significant expression of LXR target genes ABCA1 and SREBP1c.

We have synthesized and tested structural analogs of 22R and 20S in an attempt to develop more potent oxysterol analogs capable of activating LXR signaling that would have greater metabolic stability when administered systemically in animals and humans. Oxy16 is an example of such molecule that is more potent than its naturally occurring counterparts in blocking clonogenic growth of osteosarcoma cells as shown below.

### B. LXR Activation Inhibits Clonogenic Growth of Osteosarcoma Cells:

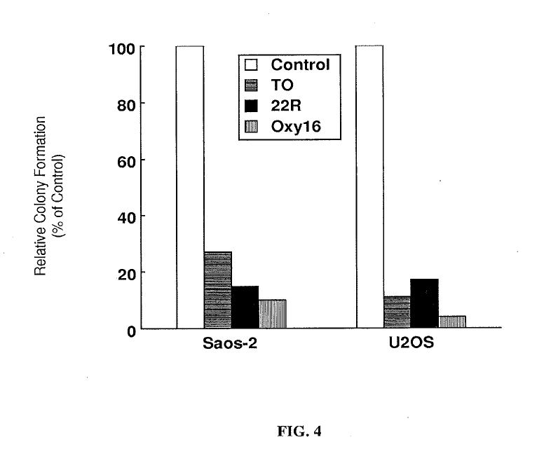

We examined whether LXR activation inhibits the clonogenic growth of human osteosarcoma cells using an anchorage-independent cell growth assay. Saos-2 and U2OS cells were seeded in standard tissue culture plates and treated for 72 hours with control vehicle or 1 μM of TO, 22R, or Oxy16. Following treatments with LXR ligands, the drugs were removed and the cells harvested and plated in methylcellulose media in non-adherent plates (Costar) and the cell colonies formed after 10 days were counted. We found that all LXR ligands resulted in significant inhibition of clonogenic growth of Saos-2 and U2OS human osteosarcoma cells (FIG. 4).

C. LXR Activation is Associated with Inhibition of Hh Target Gene Expression in Osteosarcoma Cells:

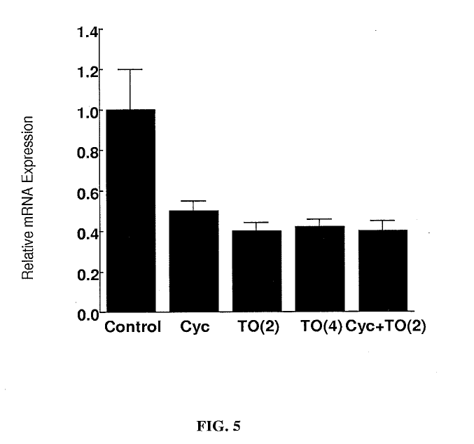

To examine whether LXR activation and inhibition of clonogenic growth in cells treated with LXR ligands are associated with inhibition of baseline Hh signaling in osteosarcoma cells, Saos-2 cells were cultured in 2% FBS and treated at 100% confluence for 72 hours with 2 or 4 τM TO, or with 4 μM cyclopamine (a hedgehog signaling pathway inhibitor that directly binds to and inhibits Smoothened). Q-RT-PCR analysis of Ptch1 mRNA expression (a gene whose expression is proportional to activity of the Hedgehog signaling pathway) showed a significant inhibition of Ptch1 expression by TO and cyclopamine (FIG. 5). There was no additive inhibitory effect when cells were treated with TO and cyclopamine together (FIG. 5) suggesting that no further inhibition of Hh signaling is achieved when cells are treated with TO and a Smoothened antagonist.

### D. LXR Activation Inhibits Clonogenic Growth of Human Multiple Myeloma Cells:

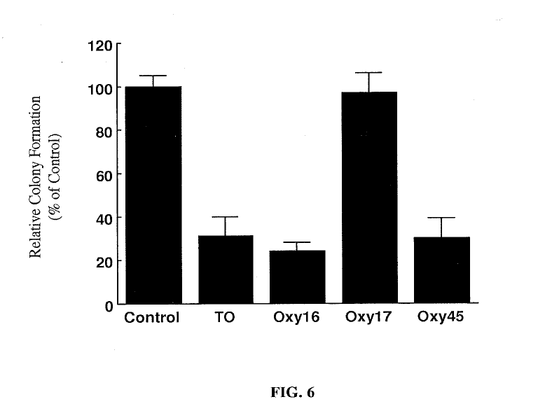

In order to examine the effect of LXR activation on the clonogenic growth of multiple myeloma cells, the human NCI-H929 multiple myeloma cell line was used. LXR activation by T0901317 (TO) or by Oxysterols Oxy16 and Oxy45, but not by Oxy17 which does not cause LXR activation, inhibited clonogenic growth of NCI-H929 cells (FIG. 6).

In addition; LXR activation inhibited clonogenic growth of multiple myeloma cancer stem cells derived from two human clinical specimens (Table 4).

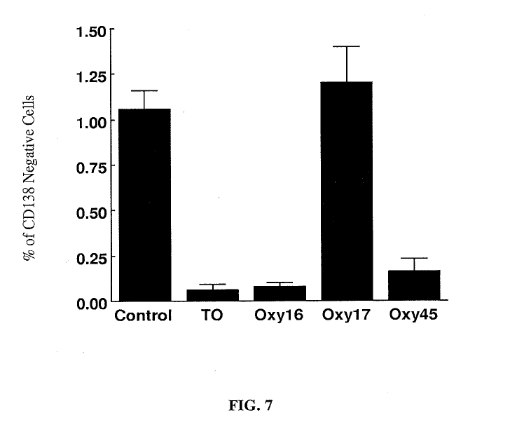

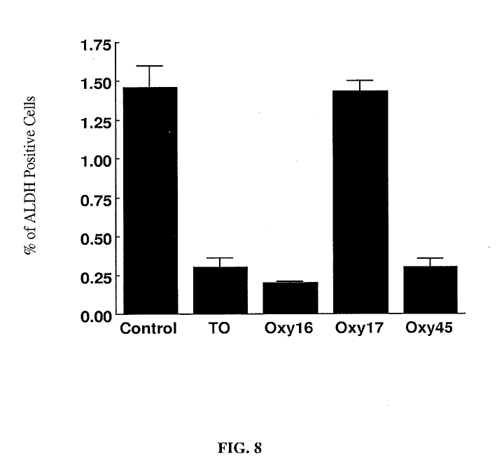

Furthermore, LXR activation by TO, Oxy16, and Oxy45, but not by Oxy17, significantly reduced the percentage of cancer stem cells in the NCI-H929 multiple myeloma cell line as evidenced by the percentage of CD138 negative and aldehyde dehydrogenase (ALDH) positive cells that are thought to represent multiple myeloma cancer stem cells (FIGS. 7, 8).

TABLE 4
Effect of LXR activation on clonogenic growth of human
primary multiple myeloma cells derived from patients. Bone
marrow mononuclear cells from patients with multiple myeloma
were depleted of CD34+ and CD138+ cells then treated with
1 μM of each compound for 96 hours followed by
assessment of clonogenic growth in methylcellulose.
Data reported as colony formation (% of control)

| Specimen # | Control | TO | Oxy16 | Oxy45 |
|---|---|---|---|---|
| 1 | 100 | 27 | 13 | 46 |
| 2 | 100 | 18 | 20 | 25 |

### 7. Further Studies on Pancreatic Cancer and Other Epithelial Neoplasms

In studies using the full LXR agonist TO901317 (TO) or naturally occurring oxysterol LXR ligand 22(R)-hydroxycholesterol (a partial agonist) (47, 48) we have found that human pancreatic cancer cells express both LXRα and LXR [symbol] and that they respond to LXR ligands, which induce the expression of LXR target genes in these cells. Furthermore, we have found that both full and partial agonists of LXRs significantly inhibit the clonogenic growth of human pancreatic cancer cells in vitro.

### Methodology and Approach:

We have screened nine human pancreatic cancer cell lines for their relative baseline LXR and Hh target gene expression, as well as their relative responsiveness to LXR ligands. We have selected three cell lines based on their varying degrees of responsiveness to LXR activation and target gene expression, with Capan-1>E3LZ10.7>L3.6p1 despite the apparently similar expression levels of LXRα and LXRβ in these cell lines. We will examine the baseline as well as Shh-induced Hh signaling in the three human pancreatic cell lines using Q-RT-PCR analysis of target gene expression and 8×-Gli luciferase reporter assays. By using TO, a full LXR agonist, as well as naturally occurring and synthetic oxysterols (partial LXR agonists) to achieve LXR activation, we will be able to distinguish any differences that might arise from using these inherently different ligands (47, 48), and we will be able to provide rationale for future in vivo translational studies of synthetic small molecule oxysterols for intervention in pancreatic cancer.

Furthermore, using a previously described modified Boyden chamber assay for invasion/migration (49), we will assess the effect of LXR activation on the invasive phenotype of these cells that would indicate their potential for cancer dissemination. Effects on proliferation will be assessed using a standard MTT assay. Since epithelial-to-mesenchymal (EM) transition has been correlated with the degree of invasiveness of pancreatic cancer cells, we will examine this phenomenon in the presence vs. absence of LXR activators. We expect that a decrease in invasiveness of the cells will correlate with inhibition of epithelial-to-mesenchymal transition evidenced by downregulation and upregulation of protein markers snail and E-cadherin, respectively (49). Moreover, since the invasiveness and resilience of pancreatic tumors to chemotherapeutic agents has been attributed to the presence of a cancer stem cell population that expresses aldehyde dehydrogenase (ALDH), we will measure the percentage of ALDH positive cells using flow cytometry (49). Inhibition of Hh signaling in pancreatic cancer cells, including the E3LZ10.7, by cyclopamine was found to significantly reduce the percentage of ALDH-expressing cells (49). Accordingly, we expect that inhibition of Hh signaling in cells upon LXR activation will also demonstrate a reduced percentage of ALDH-positive cells correlated with reduced epithelial-to-mesenchymal transition

We will expand upon the results obtained above with in vivo studies, using conventional mouse models of human pancreatic xenografts, in order to show that LXR ligands can serve as therapeutic agents for intervention with growth and dissemination of pancreatic cancer.

Accumulating evidence suggests that aberrant Hh signaling is an underlying cause of pancreatic cancer, and that inhibition of Hh signaling might prove to be an effective strategy for inhibiting pancreatic tumor formation and metastasis. Given that LXRs are known pharmacological targets for intervention in various human diseases, the use of LXR ligands for targeting pancreatic cancer cells is of great potential. We expect that these studies will confirm that the LXR agonists of the invention can target pancreatic cancer cells, without causing adverse lipogenesis.

### 8. Inhibition of Paracrine Hedgehog Signaling by LXR Agonists

As noted above, Hh signaling appears to play an important role in the initiation and progression of pancreatic cancer (26), and the inhibition of HE signaling using small molecule antagonists inhibits pancreatic cancer cells from growing in vitro and in vivo (50). More recently, it has been suggested that Hh proteins expressed by a subset of epithelial cancers, including pancreatic, colon, and ovarian cancer, promote tumor growth indirectly by activating Hh signaling in tumor stromal cells/fibroblasts that are of mesenchymal origin (51, 52). Subsequently, Hh signaling in stromal cells provides a permissive milieu for tumor cells to grow. Therefore given our previous demonstration that LXR activation inhibits Hh signaling in various stromal cells (53), it is likely that inhibition of Hh signaling by pharmacological activators of LXR may also inhibit paracrine Hh signaling in tumor fibroblasts and therefore inhibit tumor cell growth. In this Example, we examine this possibility using an in vitro model system in which Hh signaling is induced in C3H10T1/2 embryonic fibroblasts by conditioned-medium (CM) from CAPAN-1 human pancreatic cancer cells. We report that LXR activation by the non-steroidal LXR agonist, TO901317 and by oxysterols inhibit CM-induced Hh target gene expression in C3H10T1/2 cells.

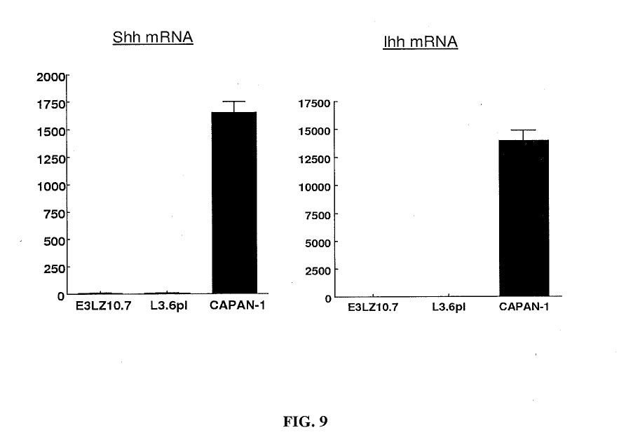

We screened several pancreatic cancer cells for the expression of Shh and Ihh and found that CAPAN-1 cells cultured to confluence in the presence of 10% FBS robustly express the mRNA for these molecules relative to L3.6p1 or E3LZ10.7 cells, with CAPAN-1>L3.6p1>E3LZ10.7 (FIG. 9). Culturing CAPAN-1 cells in 1% vs. 10% FBS had no effects on their level of mRNA expression for 11th and Shh (data not shown), and treatment of CAPAN-1 cells with the Hh pathway inhibitor cyclopamine (4 μM) or the LXR agonist TO (2-5 μM) had no effect on the expression of Ihh or Shh mRNA in these cells (data not shown).

Conditioned-Medium from CAPAN-1 Cells has Hh Activity:

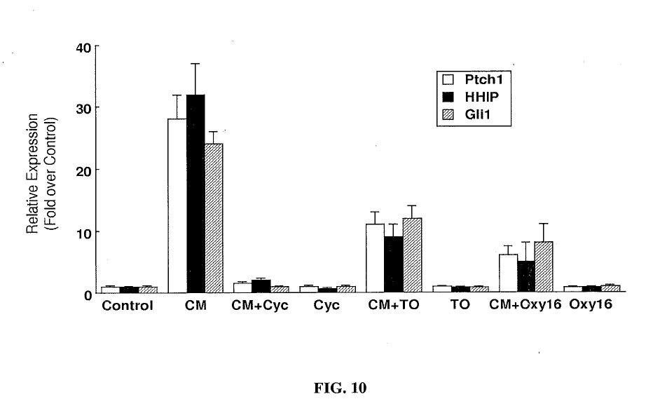

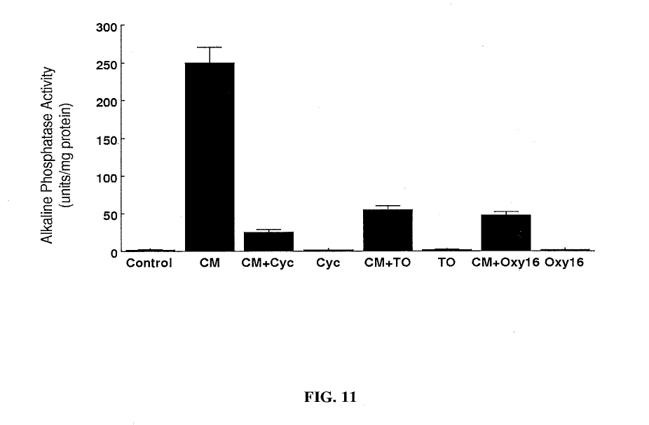

In order to assess the functional activity of Hh proteins produced by CAPAN-1 cells, we examined the ability of CM to induce Hh target gene expression in C3H10T1/2 embryonic fibroblasts. Treatment of C3H10T1/2 cells for 48 hours with CAPAN-1 CM induced robust expression of Hh target genes, Ptch1, Gli1, and HHIP in C3H10T1/2 embryonic fibroblasts, which was completely inhibited by the Hh pathway inhibitor, cyclopamine (FIG. 10). This confirmed that the expression of Ihh and Shh mRNA by CAPAN-1 cells translates into production of active Hh proteins. In addition, treatment of C3H10T1/2 cells with CM caused a significant induction of alkaline phosphatase (ALP) activity, a marker of osteogenic differentiation in these cells (FIG. 11). Similar to the inhibition of Hh target gene expression, cyclopamine also inhibited CM-induced ALP activity (FIG. 11). We and others previously reported that activation of Hh signaling induces ALP activity and osteoegnic differentiation in C3H10T1/2 cells and other multipotent stromal cells.

### LXR Agonists Inhibit CAPAN-1 CM-Induced Hh Signaling:

Next we examined whether LXR activation by LXR agonists inhibits CAPAN-1 CM-induced Hh target gene expression in fibroblastic cells. As expected, treatment of C3H10T1/2 cells with 2 M of the non-steroidal LXR agonist, TO901317 (TO), significantly induced the expression of LXR target genes, ABCA1, ABCG1, and SREBP1c after 48 hours of treatment (data not shown). Similar to the inhibitory effects of cyclopamine, treatment of C3H10T1/2 cells with TO significantly inhibited CAPAN-1 CM-induced expression of Hh target genes (FIG. 10), as well as ALP activity in these cells (FIG. 11).

As noted above, specific oxysterols are thought to be physiological ligands of LXRs that are classified as partial agonists based on their differential effects on the interaction of LXRs with co-activators and co-repressors compared to those induced by the full LXR agonist TO. We examined the effects of a synthetic oxysterol LXR agonist, Oxy16, designed and synthesized in our laboratory, on Hh signaling in C3H10T1/2 cells treated with CAPAN-1 CM. Activation of LXRs by Oxy16 was confirmed by the induction of ABCA1 and ABCG1 in C3H10T1/2 cells measured after 48 hours of treatment. Similar to the effects of TO, Oxy 16 also inhibited CM-induced Hh target gene expression (FIG. 10) and ALP activity (FIG. 11) in C3H10T1/2 cells (FIG. 11). The inhibitory effects of Oxy 16 used at 5 μM were similar to those of TO at 2 μM. In addition, another oxysterol LXR agonist 22(R)-hydroxycholesterol also inhibited CM-induced Hh signaling, whereas 22(S)-hydroxycholesterol, which is not an LXR agonist, did not have similar inhibitory effects.

### 9. In Vivo Demonstrations that Oxysterols of the Invention Function as Disclosed Herein

1) Studies on cell proliferation. Tumor cells or excised human tumors are used as xenografts in nude mice in order to induce tumor formation. i.v. and/or i.p. and/or subcut and/or IM and/or orally. Administration of the LXR agonists of the invention are expected to decrease, for example, one or more of the following indices: tumor cell engraftment, tumor growth, tumor size, tumor burden, or serologic markers of tumor formation if any (e.g. PSA in the case of prostate cancer tumors, CA125 in the case of ovarian tumors).
2) Studies on the prevention and reversal of atherosclerosis. LXR agonists of the invention are administered to various mouse models of atherosclerosis, including, e.g., C57BL/6 mice on a high fat diet, ApoE null mice on a regular chow diet, LDL receptor null mice on a chow diet. All these mice develop dyslipidemia including increased total cholesterol, increased LDL cholesterol, increased triglycerides, decreased HDL, and would develop atherosclerotic lesions in the arteries. Administration of LXR agonists would be expected to correct some or all of these disorders and result in reduced lesion formation.
3) Studies on the treatment or prevention of Alzheimer's disease. LXR ligands of the invention are administered to mouse models of Alzheimer's disease and then the amount of beta amyloid deposition in the brains of these mice is measured compared to placebo treated mice. Mice receiving LXR ligands are expected to perform better than those receiving placebo in standard assays of cognitive function in rodents.

### Treatment by Targeted Delivery of Liver X Receptor Agonist

In order to minimize potential side effects, and maximize the concentration of liver X receptor agonist to which cancer or tumor cells are exposed, a method of treatment may use a targeted approach to deliver Hedgehog-inhibiting LXR agonist directly to the cancer or tumor cells. For example, mechanical means can be used to deliver the Hedgehog-inhibiting LXR agonist to the cancer cells. For example, a catheter can be inserted into or next to a tumor or region of cancerous cells, and the Hedgehog-inhibiting LXR agonist administered at a controlled rate. A controlled release device can be implanted into or next to a tumor or region of cancerous cells, so that the Hedgehog-inhibiting LXR agonist is released at a controlled rate. Alternatively, a biomolecular targeting approach can be used to deliver Hedgehog-inhibiting LXR agonist to tumor or cancer cells. For example, stem cells tend to concentrate near proliferating cancer or tumor cells.

### Administration of Liver X Receptor Agonists

Hedgehog-inhibiting liver X receptor (LXR) agonists can be administered by any one of or a combination of several routes. For example, compositions of the invention can be administered orally, injected, e.g., injected intravenously or intraperitonealy or intramuscularly, or administered topically. For research purposes, the route of administration selected by the researcher can depend on the topic of study. For therapeutic purposes, the route of administration to a subject selected by the clinician can depend on, for example, the disease state, the extent of the disease, the general physical condition of the subject, and a number of other factors. For example, a Hedgehog-inhibiting LXR agonist can be administered topically to the site of a basal cell carcinoma to treat this disease.

We will test oxysterols of the invention for their ability to inhibit the growth and dissemination of tumor cells in a variety of human and other animal cancers, using conventional methods such as those described herein. It is expected that an oxysterol of the invention that inhibits Hedgehog signaling, through activation of LXR signaling and/or other molecular mechanism, will inhibit the growth and dissemination of tumor cells in a variety of human and other animal cancers, including those discussed herein.

We will examine the efficacy of oxysterols of the invention for inhibiting tumor growth and/or metastasis, using conventional experimental models in which human tumor xenografts are placed in immunodeficient mice. We expect that the administration of oxysterols to these mice will inhibit growth and/or metastasis of the xenografts. Without wishing to be bound by any particular mechanism, it is suggested that this inhibition will be achieved through activation of LXR signaling, and/or inhibition of Hedgehog signaling, and/or through other mechanisms.

We will test oxysterols of the invention for their ability to serve as preventative as well as therapeutic agents for cancers, as well as a variety of other disorders that arise from unregulated cellular proliferation, using conventional testing procedures. It is expected that the administration of the oxysterols of the invention will serve as a preventative as well as a therapeutic strategy for intervention in cancers, as well as in other disorders that arise from unregulated cellular proliferation. We will also test for the ability of oxysterols of the invention to act as preventative of therapeutic agents for the other suitable disease conditions discussed herein, using conventional methods. It is expected that the oxysterols will act as predicted.

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever. The entire disclosure of all applications, patents, and publications cited above, including U.S. Provisional application 61/305,046, filed Feb. 16, 2010, are hereby incorporated by reference in their entirety.

### REFERENCES

1. Mullor J L, Sanchez P, Altaba A R. Pathways and consequences: Hedgehog signaling in human disease. Trends in Cell Biol 12:562-569; 2002.
2 Ehlen H W A, Buelens L A, Vortkamp A. Hedgehog signaling in skeletal development. Birth Defects Res 78:267-279; 2006.
3. Cooper M K, Wassif C A, Krakowiak P A, Taipale J, Gong R, Kelley R I, Porter N D, Beachy P A. A defective response to Hedgehog signaling in disorders of cholesterol biosynthesis. Nat Genet 33:508-513; 2003.
4. Maeda Y, Nakamura E, Nguyen M T, Suva L J, Swain F L, Razzaque M S, Mackem S, Lanske B. Indian Hedgehog produced by postnatal chondrocytes is essential for maintaining a growth plate and trabecular bone. Proc Nat Acad Sci 104:6382-6387; 2007.
5. Beachy P A, Karhadkar S S, Berman D M. Tissue repair and stem cell renewal in carcinogenesis. Nature 432:324-330; 2004.
6. Bale A E. Hedgehog signaling and human disease. Annu Rev Genomics Hum Genet 3:47-65; 2002.
7. Rubin L L, de Sauvage F J. Targeting the Hedgehog pathway in cancer. Nature Rev 5:1026-1033; 2006.
8. Scales S J, de Sauvage F J. Mechanisms of Hedgehog pathway activation in cancer and implications for therapy. Trends Pharmacol Sci 30:303-312; 2009.
9. Von Hoff D D, LoRusso P M, Rudin C M, Reddy J C, Yauch R L, Tibes R, Weiss G J, Borad M J, Hann C L, Brahmer J R, Mackey H M, Lum B L, Darbonne W C, Marsters J C, de Sauvage F J. Inhibition of the Hedgehog pathway in advanced basal-cell carcinoma. N Engl J Med 361:1164-1172; 2009.
10. Rudin C M, Hann C L, Laterra J, Yauch R L, Callahan C A, Fu L, Holcomb T, Stinson J, Gould S E, Coleman B, LoRusso P M, Von Hoff D D, de Sauvage F J. Treatment of medulloblatoma with Hedgehog pathway inhibitor GDC-0449. N Engl J Med 361:1173-1178; 2009.
11. Edwards P A, Kennedy M A, Mak P A. LXRs; Oxysterol-activated nuclear receptors that regulate genes controlling lipid homeostasis. Vasc Pharm 38:249-256; 2002.
12. Kalaany N Y, Mangelsdorf D J. LXRs and FXR: The Yin and Yang of cholesterol and fat metabolism. Annu Rev Physiol 68:159-191; 2006.
13. Edwards P A, Kast H R, Anisfeld A M. BAREing it all: the adoption of LXR and FXR and their roles in lipid homeostasis. J Lipid Res 43:2-12; 2002.
14. Joseph S B, Castrillo A, Laffitte B A, Mangelsdorf D J, Tontonoz P. Reciprocal regulation of inflammation and lipid metabolism by liver X receptors. Nat Med 9:213-219; 2003.
15. Raghaw R, Yellaturu C, Deng X, Park E A, Elam M B. SREBPs: the crossroads of physiological and pathological lipid homeostasis. Trends Endocrinol Metab 19:65-73; 2008.
16. Bengoechea-Alonso M T, Ericsson J. SREBP in signal transduction: cholesterol metabolism and beyond. Curr Opin Cell Bio 19:215-222; 2007.
17. Shimano H. SREBPs: physiology and pathophysiology of the SREBP family. FEBS J 276:616-621; 2008.
18. Takahashi K, Kimura Y, Nagata K, Yamamoto A, Matsuo M, Ueda K. ABC proteins: key molecules for lipid homeostasis. Med Mol Morphol 38:2-12; 2005.
19. Rangwala F, Omenetti A, Diehl A M. Cancer stem cells: repair gone awry? J Oncoloogy 2011:1-11; 2011 [Epub ahead of print].
20. Hirota M, Setoguchi T, Sasaki H, Matsunoshita Y, Gao H, Nagao H, Kunigou O, Komiya S. Smoothened as a new therapeutic target for human osteosarcoma. Molecular Cancer 9:1-14; 2010.
21. Lum L, Beachy P A. The Hedgehog response network: sensors, switches, and routers. Science 304:1755-1759; 2004.
22. Bijlsma M F, Spek C A, Peppelenbosch M P. Hedgehog: an unusual signal transducer. Bioessays 26:387-394; 2004.
23. Riobo N A, Lu K, Ai X, Haines G M, Emerson C P. Phosphoinositide 3-kinase and Akt are essential for sonic Hedgehog signaling. Proc Nat Acad Sci 103:4505-4510; 2006.
24. Wendler F, Franch-Marro X, Vincent J P. How does cholesterol affect the way Hedgehog work? Development 133:3055-3061; 2006.
25. Porter J A, Young K E, Beachy P A. Cholesterol modification of Hedgehog signaling proteins in animal development. Science 274:255-259; 1996.
26. Scales S J, de Sauvage F J. Mechanisms of Hedgehog pathway activation in cancer and implications for therapy.
27. Von Hoff D D, LoRusso P M, Rudin C M, Reddy J C, Yauch R L, Tibes R, Weiss G J, Borad M J, Hann C L, Brahmer J R, Mackey H M, Lum B L, Darbonne W C, Marsters J C, de Sauvage F J Inhibition of the Hedgehog pathway in advanced basal-cell carcinoma. New Engl J Med 361:1164-1172; 2009.
28. Liao X, Siu M, Au C, Wong E, Chan H, Ip P, Ngan Y, Cheung N Y. Aberrant activation of hedgehog signaling pathway in ovarian cancers: effect on prognosis, cell invasion and differentiation. Carcinogenesis 30:131-140; 2009.
29. Barginear M F, Leung M, Budman D R. The hedgehog pathway as a therapeutic target for treatment of breast cancer. Breast Cancer Res Treat 116:239-246; 2009.
30. Shaw A, Gipp J, Bushman W. The Sonic Hedgehog pathway stimulates prostate tumor growth by paracrine signaling and recapitulates embryonic gene expression in tumor myofibroblasts. Oncogene 28:4480-4490; 2009.
31. Peacock C D, Wang Q, Gesell G S, Corcoran-Schwartz I M, Jones E, Kim J, Devereux W L, Rhodes J T, Huff C A, Beachy P A, Watkins D N, Matsui W. Hedgehog signaling maintains a tumor stem cell compartment in multiple myeloma. Proc Natl Acad Sci USA 104:4048-4053; 2007.
32. Hegde G V, Peterson K J, Emanuel K, Mittal A K, Joshi A D, Dickinson J D, Kollessery G J, Bociek R G, Bierman P, Vose J M, Weisenburger D D, Joshi S S. Hedgehog-induced survival of B-cell chronic lymphocytic leukemia cells in a stromal cell microenvironment: a potential new therapeutic target. Mol Cancer Res 6:1928-1936; 2008.
33. Edwards P A, Kennedy M A, Mak P A. LXRs; Oxysterol-activated nuclear receptors that regulate genes controlling lipid homeostasis. Vasc Pharm 38:249-256; 2002.
34. Kalaany N Y, Mangelsdorf D J. LXRs and FXR: the yin and yang of cholesterol and fat metabolism. Annu Rev Physiol 68:159-191, 2006.
35. Edwards P A, Kast H R, Anisfeld A M. BAREing it all: the adoption of LXR and FXR and their roles in lipid homeostasis. J Lipid Res 43:2-12; 2002.
36. Crisafulli C, Mazzon E, Paterniti I, Galuppo M, Bramanti P, Cuzzocrea S. Effects of Liver x receptor agonist treatment on signal transduction pathways in acute lung inflammation. Respir Res 11:1-15; 2010.
37. Vedin L, Lewandowski S A, Parini P, Gustafsson J, Steffensen K R. The oxysterol receptor LXR inhibits proliferation of human breast cancer cells. Carcinogenesis 30:575-579; 2009.
38. Pommier A J C, Alves G, Viennois E, Bernard S, Communal Y, Sion B, Marceau G, Damon C, Mouzat K, Caira F, Baron S, Lobaccaro J M A. Liver X Receptor activation downregulates AKT survival signaling in lipid rafts and induces apoptosis of prostate cancer cells. Oncogene 29:2712-2723; 2010.
39. Scoles D R, Xu X, Wang H, Tran H, Taylor-Harding B, Li A, Karlan B Y. Liver X receptor agonist inhibits proliferation of ovarian carcinoma cells stimulated by oxidized low density lipoprotein. Gynecol Oncol 116:109-116 (2010).
40. Joseph S B, McKillingin E, Pei L, Watson M A, Collins A R, Laffitte B A, Chen M, Hoh G, Goodman J, Nagger G N, Tran 0.1, Tippin T K, Wang X, Lusis A J, Hsueh W A, Law R E, Collins J L, Willson T M, Tontonoz P. Synthetic LXR ligand inhibits the development of atherosclerosis in mice. Proc Nat Acad Sci 99:7604-7609; 2002.
41. Radder D J. Liver X receptor and farnesoid X receptor as therapeutic targets. Am J Cardiol 100:15N-19N; 2007.
42. Laffitte B A, Chao L C, Li J, Walczak R, Hummasti S, Joseph S B, Castrillo A, Wilpitz D C, Mangelsdorf D J, Collins J L, Saez E, Tontonoz P. Activation of liver X receptor improves glucose tolerance through coordinate regulation of glucose metabolism in liver and adipose tissue. Proc Natl Acad Sci USA 100:5419-5424; 2003.
43. Zelcer N, Khanlou N, Clare R, Jiang Q, Reed-Geaghan E G, Landreth G E, Vinters H V, Tontonoz P. Attenuation of neuroinflammation and Alzheimer's disease pathology by liver X receptors. Proc Nati Acad Sci USA 104:10601-10606.
44. Watanabe B, Nakagawa Y, Ogura T, Miyagawa H. Stereoselective synthesis of (22R)- and (22S)-castasterone/ponasterone A hybrid compounds and evaluation of their molting hormone activity. Steroids 69:483-493; 2004.
45. Drew J, Letellier M, Morand P, Szabo A G. Synthesis from pregnenolone of fluorescent cholesterol analogue probes conjugated unsaturation in the side chain J Org Chem 52:4047-4052; 1987.
46. De la Rosa M A, Velarde E, Guzman A. Cross-coupling reactions of monosubstituted acetylenes and aryl halides catalyzed by palladium on charcoal. Synth Commun 20(13):2059-2064; 1990.
47. Albers M, Blume B, Schlueter T, Wright M B, Kober I, Kremoser C, Deuschle U, Koegl M. A novel principle for partial agonism of liver X receptor ligands. J Biol Chem 281:4920-4930; 2006.
48. Phelan C A, Weaver J M, Steger D J, Joshi S, Maslany J T, Collins J L, Zuercher W J, Willson T M, Walker M, Jaye M, Lazar M A. Selective partial agonism of liver X receptor a is related to differential corepressor recruitement. Mol Endocrinol 22: 2241-2249; 2008.
49. Feldmann G, Dhara S, Fendrich V, Bedja D, Beaty R, Mullendore M, Karikari C, Alvarez H, Iacobuzio-Donahue C, Jimeno A, Gabrielson K L, Matsui W, Maitra A. Blockade of IIedgehog signaling inhibits pancreatic cancer invasion and metastasis: A new paradigm for combination therapy in solid tumors. Cancer Res 67:2187-2196; 2007.
50. Feldmann G, Fendrich V, McGovern K, Bedja D, Bisht S, Alvarez H, Koorstra J M, Habbe N, Karikari C, Mullendore M, Gabrielson K L, Sharma R, Matsui W, Maitra A. An orally bioavailable small molecule inhibitor of Hedgehog signaling inhibits tumor initiation and metastasis in pancreatic cancer. Mol Cancer Ther 7:2725-2735; 2008.
51. Yauch R L, Gould S E, Scales S J, Tang T, Tian H, Ahn C P, Marshall D, Fu L, Januario T, Kallop D, Nannini-Pepe M, Kotkow K, Marsterns J C, Rubin L L, de Sauvage F J. A paracrine requirement for Hedgehog signaling in cancer. Nature 455:406-410; 2008.
52. Tian H, Callahan C A, DuPree K J, Darbonne W C, Ahn C P, Scales S J, de Sauvage F J. Hedgehog signaling is restricted to the stromal compartment during pancreatic carcinogenesis. Proc Nat Acad Sci 106:4254-4259; 2009.
53. Kim W K, Meliton V, Park K W, Hong C, Tontonoz P, Niewiadomski P, Waschek J A, Tetradis S, Parhami F. Negative regulation of Hedgehog signaling by liver X receptors. Mol Endocrinol 23: 1532-1543; 2009.

### OTHER REFERENCES OF INTEREST

Jiang J, Hui C. Hedgehog signaling in development and cancer. Develop Cell 15:801-812; 2008.
Zelcer N, Tontonoz P. Liver X receptors as integrators of metabolic and inflammatory signaling. J Clin Invest 116:607-614; 2006.
Gill 5, Chow R, Brown A J. Sterol regulators of cholesterol homeostasis and beyond: The oxysterol hypothesis revisited and revised. Prog Lipid Res 47:391-404; 2008.
Vedin L, Lewandowski S A, Parini P, Gustafsson J, Steffensen K R. The oxysterol receptor LXR inhibits proliferation of human breast cancer cells. Carcinogenesis 30:575-579; 2009.
Chuu C, Hiipakka R A, Kokontis J M, Fukuchi J, Chen R, Liao S Inhibition of tumor growth and progression of LNCaP prostate cancer cells in athymic mice by androgen and liver X receptor agonist. Cancer Res 66:6482-6486; 2006.
Geyeregger R, Shehata M, Zeyda M, Kiefer F W, Stuhlmeier K M, Porpaczy E, Zlabinger G J, Jager U, Stulnig T M. Liver X receptors interfere with cytokine-induced proliferation and cell survival in normal and leukemic lymphocytes. J Leukoc Biol; 2009 [Epub ahead of print]. Thayer S P, di Magliano M P, Heiser P W, Nielsen C M, Roberts D J, Lauwers G Y, Qi Y P, Gysin 5, Fernandez del-Castillo C, Yajnik V, Antoniu B, McMahon M, Warshaw A L, Hebrok M.
Hedgehog is an early and late mediator of pancreatic cancer tumorigenesis. Nature 425:851-856; 2003.
Bailey J M, Mohr A M, Hollingsworth M A. Sonic Hedgehog paracrine signaling regulates metastasis and lymphangiogenesis in pancreatic cancer. Oncogene; 2009 [Epub ahead of print].
Olive K P, Jacobetz M A, Davidson C J, Gopinathan A, McIntyre D, IIoness D, Madhu B, Goldgraben M A, Allard D, Frese K K, DeNicola G, Feig C, Combs C. Inhibition of Hedgehog signaling enhances delivery of chemotherapy in a mouse model of pancreatic cancer. Science 324:1457-1461; 2009.
Ehlen H W A, Buelens L A, Vortkamp A. Hedgehog signaling in skeletal development. Birth Defects Res 78:267-279; 2006.
Bijlsma M F, Peppelenbosch M P, Spek C A. Hedgehog morphogen in cardiovascular disease. Circulation 114:1985-1991; 2006.
Jiang J, Hui C. Hedgehog signaling in development and cancer. Develop Cell 15:801-812; 2008.
Naik S U, Wang X, Da Silva J S, Jaye M, Macphee C H, Reilly M P, Billheimer J T, Rothblat G H, Rader D J. Pharmacological activation of liver X receptors promotes reverse cholesterol transport in vivo. Circulation 113:90-97; 2006.
Joseph S B, Castrillo A, Laffitte B A, Mangelsdorf D J, Tontonoz P. Reciprocal regulation of inflammation and lipid metabolism by liver X receptors. Nat Med 9:213-219; 2003.
Zelcer N, Tontonoz P. Liver X receptors as integrators of metabolic and inflammatory signaling. J Clin Invest 116:607-614; 2006.
Olive K P, Jacobetz M A, Davidson C J, Gopinathan A, McIntyre D, Honess D, Madhu B, Goldgraben M A, Caldwell M E, Allard D, Frese K K, DeNicola G, Feig C, Combs C, et al. Inhibition of Hedgehog signaling enhances delivery of chemotherapy in a mouse model of pancreatic cancer. Science 324:1457-1461; 2009.
Theunissen J, de Sauvage F J. Paracrine Hedgehog signaling in cancer. Cancer Res 69:6007-6010; 2009.
Tall A R. Cholesterol efflux pathways and other potential mechanisms involved in the athero-protective effect of high density lipoproteins. J Internal Med 263:256-273; 2008.
Baranowski M. Biological role of liver X receptors. J Physiol Pharmacol 59 Suppl 7:31-55; 2008.
Kansara M, Thomas D M. Molecular pathogenesis of osteosarcoma. DNA Cell Biol 26:1-18; 2007.
Ta H T, Dass C R, Choong P F M, Dunstan D E. Osteosarcoma treatment: state of the art. Cancer Metastasis Rev 28:247-263; 2009.
St-Jacques B, Hammerschmidt M, McMahon A P. Indian Hedgehog signaling regulates proliferation and differentiation of chondrocytes and is essential for bone formation. Genes Dev 13:2072-2086; 1999.
Dlugosz A A, Talpaz M. Following the Hedgehog to new cancer therapies. N Engl J Med 361:1202-1205; 2009.
Stein U, Eder C, Karsten U, Haensch W, Walther W, Schlag P M. Gli1 gene expression in bone and soft tissue sarcomas of adult patients correlates with tumor grade. Cancer Res 59:1890-1895; 1999:
Chisholm J W, Hong J, Mills S A, Lawn R M. The LXR ligand TO901317 induces severe lipogenesis in the db/db diabetic mice. J Lipid Res 44:2039-2048; 2003.
Benassi M S, Chiechi A, Ponticelli F, Pazzaglia L, Gamberi G, Zanella L, Manara M C, Perego P, Ferrari S, Picci P. Growth inhibition and sensitization to cisplatin by zolendronic acid in osteosarcoma cells. Cancer Lett 250:194-205; 2007.
Mojcicka O, Jamroz-Wisniewska A, Horoszewicz K, Beltowski J. Liver X receptors (LXRs). Part I: Structure, function, regulation of activity, and role in lipid metabolism. Postepy Hig Med Dosw (Online) 61:736-759; 2007.
Caspary T, Larkins C E, Anderson K V. The graded response to sonic Hedgehog depends on cilia architecture. Dev Cell 12:767-778; 2007.
Choe S S, Choi A H, Lee J, Kim J H, Chung J, Park J, Lee K, Park K, Lee I, Kim J B. Chronic activation of liver X receptor induces β-cell apoptosis through hyperactivation of lipogenesis. Diabetes 56:1534-1543; 2007.
Seres L, Cserepes J, Elkind N B, Torocsik D, Nagy L, Sarkadi B, Homolya L. Functional ABCG1 expression induces apoptosis in macrophages and other cell types. Biochimica Biophysica Acta 1778:2378-2387; 2008.
Uno S, Endo K, Jeong Y, Kawana K, Miyachi H, Hashimoto Y, Makishima M. Suppression of β-catenin signaling by liver X receptor ligands. Biochem Pharmacol 77:186-195; 2009.

1. A pharmaceutical composition for reducing the proliferation or metastatic activity of a cell or tissue, wherein the pharmaceutical composition comprises a compound represented by Formula II and a pharmaceutically acceptable carrier: 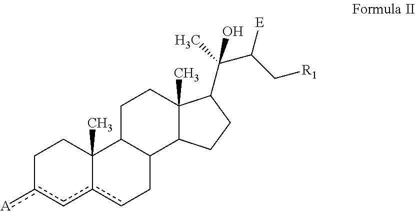
wherein A is selected from the group consisting of hydrogen, hydroxy, or oxygen, [symbol]
is a single or a double bond,
E is hydrogen or hydroxy, and
R₁ is

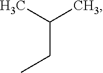

2. The pharmaceutical composition of claim 1, wherein the compound represented by Formula II comprises one or more of Oxy 16, Oxy30, Oxy 31, Oxy35, or Oxy37.

3. The pharmaceutical composition of claim 1, wherein the cell or tissue is in vitro.

4. The pharmaceutical composition of claim 1, wherein the cell or tissue is in an animal.

5. The pharmaceutical composition of claim 4, wherein the animal is a human.

6. The pharmaceutical composition of claim 1, wherein the proliferation or metastatic activity is of a cell or tissue in a cancer.

7. The pharmaceutical composition of claim 1, wherein the proliferation or metastatic activity is of a cell or tissue in a tumor.

8. The pharmaceutical composition of claim 1, wherein the proliferation or metastatic activity is of a cell or tissue in basal cell carcinoma, melanoma, multiple myeloma, leukemia, stomach cancer, bladder cancer, prostate cancer, ovarian cancer, or bone cancer.

9. The pharmaceutical composition of claim 1, wherein the reduction or the proliferation or metastatic activity is a reduction of the prevalence of cancer stem cells in a subject.

10. The pharmaceutical composition of claim 1, which further comprises an additional therapeutic agent for reducing the proliferation or metastatic activity of a cell or tissue.

11. A pharmaceutical composition, comprising one or more of Oxy 30, Oxy35, or Oxy37, and a pharmaceutically acceptable carrier:

12. (canceled)

13. The pharmaceutical composition of claim 11 for reducing the proliferation or metastatic activity of a cell or tissue.

14. The pharmaceutical composition of claim 13, wherein the cell or tissue is in vitro.

15. The pharmaceutical composition of claim 13, wherein the cell or tissue is in an animal.

16. The pharmaceutical composition of claim 15, wherein the animal is a human.

17. The pharmaceutical composition of claim 13, wherein the proliferation or metastatic activity is of a cell or tissue in a cancer.

18. (canceled)

19. The pharmaceutical composition of claim 13, wherein the proliferation or metastatic activity is of a cell or tissue in basal cell carcinoma, melanoma, multiple myeloma, leukemia, stomach cancer, bladder cancer, prostate cancer, ovarian cancer, or bone cancer.

20. The pharmaceutical composition of claim 13, wherein the reduction or the proliferation or metastatic activity is a reduction of the prevalence of cancer stem cells in a subject.

21. The pharmaceutical composition of claim 13, which further comprises an additional therapeutic agent for reducing the proliferation or metastatic activity of a cell or tissue.

22. The pharmaceutical composition of claim 11 for stimulating a liver X receptor (LXR) and/or inhibiting Hedgehog (Hh) signaling in a cell or tissue.

23.-37. (canceled)
